(19) United States
(12) Patent Application Publication
(10) Pub. No.: US 2025/0255721 A1
(43) Pub. Date: Aug. 14, 2025

(54) MEDICAL DEVICE AND IMPLANT SYSTEM

(71) Applicant: United InnoMed (Shanghai) Limited, Shanghai (CN)

(72) Inventor: Li WANG, Shanghai (CN)

(73) Assignee: United InnoMed (Shanghai) Limited, Shanghai (CN)

(21) Appl. No.: 19/191,281

(22) Filed: Apr. 28, 2025

Related U.S. Application Data
(63) Continuation-in-part of application No. PCT/CN2023/126911, filed on Oct. 26, 2023.
(63) Continuation-in-part of application No. PCT/CN2023/126908, filed on Oct. 26, 2023.
(63) Continuation-in-part of application No. PCT/CN2023/126897, filed on Oct. 26, 2023.

(30) Foreign Application Priority Data
Oct. 27, 2022 (CN) 202211327896.5
Oct. 27, 2022 (CN) 202211327911.6
Oct. 27, 2022 (CN) 202211328166.7
Oct. 27, 2022 (CN) 202211330036.7

(51) Int. Cl. A61F 2/24; A61B 5/0215; A61F 2/00; A61F 2/48

(52) U.S. Cl. CPC A61F 2/246; A61B 5/0215; A61F 2/482; A61F 2002/0081; A61F 2220/0008; A61F 2250/0002; A61F 2250/008

(57) ABSTRACT

Disclosed is a medical device for improving a function of a valve of a patient. This valve includes a plurality of leaflets. The medical device includes a pad configured to be positioned among the plurality of leaflets of the valve, to make the valve periodically open and close by cooperating with the plurality of leaflets. Via the pad located among the plurality of leaflets, the medical device provided by the present disclosure is able to improve a closure condition of the valve, so that the valve is able to close properly, and thus regurgitation of blood is effectively prevented or reduced.

### CROSS-REFERENCE TO RELATED APPLICATIONS

The present application is a continuation-in-part application of International Application No. PCT/CN2023/126911, filed on Oct. 26, 2023, International Application No. PCT/CN2023/126908, filed on Oct. 26, 2023, and International Application No. PCT/CN2023/126897, filed on Oct. 26, 2023. International Application No. PCT/CN2023/126911 claims priority to Chinese Application No. 202211327911.6, filed on Oct. 27, 2022, and Chinese Application No. 202211328166.7, filed on Oct. 27, 2022. International Application No. PCT/CN2023/126908 claims priority to Chinese Application No. 202211327896.5, filed on Oct. 27, 2022. International Application No. PCT/CN2023/126897 claims priority to Chinese Application No. 202211330036.7, filed on Oct. 27, 2022. The entire contents of all the above applications are incorporated herein by reference.

### TECHNICAL FIELD

The present disclosure relates to the field of medical apparatus and instruments technologies, and in particular, to a medical device and an implant system for repairing a heart valve of a patient.

### BACKGROUND

A heart valve refers to a valve located between an atrium and a ventricle or a valve located between the ventricle and an artery. The valve plays a critical role in the heart' ceaseless blood circulation. After blood flows through, the valve closes to prevent blood regurgitation.

Various structural factors may affect proper closure of the heart valve, and thereby inducing the blood regurgitation. Taking mitral valve as an example, if the mitral valve fails to close properly, the blood may flow from the left ventricle to the left atrium through the mitral valve during systole, and thereby impairing a patient's health. A repair device for repairing the heart valve provided by related technologies has a structure similar to a clip. This medical device improves the condition of valve closure insufficiency by clipping onto a pair of leaflets of the valve.

However, such repair device has may defects. For instance, an effective area for the blood to flow through the valve is reduced due to such repair device, and so that another issue may be caused-stenosis. Additionally, as this repair device clips the pair of leaflets together, the pair of leaflets are prevented from naturally coaptating and separating during a cardiac cycle. As time goes on, this may cause problems to the structure and function of the pair of leaflets, then their associated tissues, and even the entire valve.

### SUMMARY

In view of the above, in a first aspect, the present disclosure provides a medical device. The medical device provided by the present disclosure is used for improving the function of a patient's heart valve. This medical device includes a pad, the pad is configured to be capable of being positioned among a plurality of leaflets of the valve, to make the valve periodically open and close by cooperating with the leaflets.

In a second aspect, the present disclosure provides a medical device for repairing a heart valve of a patient. The medical device is adapted to be implanted in the patient's body to repair the valve. This medical device includes a pad and a control unit. The pad is configured to be located among the plurality of leaflets of the valve, to make the valve periodically open and close by cooperating with the plurality of leaflets. The pad includes an adjustment mechanism. The control unit is configured to control the adjustment mechanism to adjust a dimension of the pad.

In a third aspect, the present disclosure provides a cardiac implant adapted to be implanted in a patient's heart. The heart includes a first chamber, a second chamber and a valve. The valve is configured to periodically allow blood to flow from the first chamber to the second chamber. The cardiac implant includes a treatment device and at least one sensor. The treatment device is used for repairing the function of the valve to prevent the blood from flowing back from the second chamber to the first chamber, and the at least one sensor is attached to the treatment device and configured to sense a physiological information of the patient.

In a fourth aspect, the present disclosure further provides an implant system. The implant system includes the cardiac implant according to the above aspect and an external device. The at least one sensor includes a wireless transmission unit, and the external device includes a wireless reception unit configured to receive a pressure value information sent from the wireless transmission unit of the sensor.

### BRIEF DESCRIPTION OF THE DRAWINGS

In order to more clearly illustrate technical solutions of embodiments of the present disclosure, the drawings to be used in the embodiments will be introduced briefly below.

It should be understood that, the drawings below merely show some embodiments of the present disclosure, but not all the embodiments.

It should be understood that, the same or similar reference numerals are used in the drawings for representing the same or similar elements.

It should be understood that, the drawings are merely illustrative, and dimensions and proportions of the elements in the drawings are not necessarily accurate.

FIG. 1 is a schematic structural diagram of a medical device according to an embodiment of the present disclosure.

FIG. 2 is a schematic structural diagram of a medical device according to another embodiment of the present disclosure.

FIG. 3A and FIG. 3B are schematic structural diagrams of a pad of the medical device of FIG. 2.

FIG. 4 is an axonometric schematic structural diagram of the medical device in FIG. 2.

FIG. 5 is a schematic structural diagram of a pad of a medical device according to another embodiment of the present disclosure.

FIG. 6 is a schematic structural diagram of a medical device according to yet another embodiment of the present disclosure.

FIG. 7 is a schematic structural diagram of a medical device according to still another embodiment of the present disclosure.

FIG. 8A and FIG. 8B are schematic structural diagrams of a pad of the medical device of FIG. 7.

FIG. 9 is a schematic structural diagram of a medical device according to further another embodiment of the present disclosure.

FIG. 10 is a schematic structural diagram of a medical device according to yet a further embodiment of the present disclosure.

FIG. 11 is a schematic structural diagram of a medical device according to an embodiment of the present disclosure.

FIG. 12A and FIG. 12B are schematic structural diagrams illustrating a pad, a control system, a sensor, and the like of the medical device of FIG. 11.

FIG. 13A and FIG. 13B are schematic structural diagrams of at least a portion of a pad of a medical device according to another embodiment of the present disclosure.

FIG. 14 is a schematic structural diagram of at least a portion of a medical device according to yet another embodiment of the present disclosure.

FIG. 15 is a schematic structural diagram of at least a portion of a medical device according to still another embodiment of the present disclosure.

FIG. 16 is a schematic structural diagram of at least a portion of a medical device according to further another embodiment of the present disclosure.

FIG. 17 is a schematic structural diagram of a cardiac implant according to an embodiment of the present disclosure.

FIG. 18 is a schematic structural diagram illustrating the cardiac implant of FIG. 17 located in a patient's heart.

FIG. 19A is a schematic structural diagram illustrating a cardiac implant according to another embodiment of the present disclosure that is located in a patient's heart.

FIG. 19B is a schematic structural diagram illustrating a cardiac implant according to yet another embodiment of the present disclosure that is located in a patient's heart.

FIG. 20 is a schematic structural diagram illustrating a cardiac implant according to still another embodiment of the present disclosure that is located in a patient's heart.

FIG. 21 is a schematic structural diagram illustrating a cardiac implant according to further another embodiment of the present disclosure that is located in a patient's heart.

FIG. 22 is a schematic structural diagram of an implant system according to an embodiment of the present disclosure.

### DETAILED DESCRIPTION OF THE EMBODIMENTS

To make a person skilled in the art better understand technical solutions of the present disclosure, the technical solutions of implementations of the present disclosure will be described below with reference to the drawings. Apparently, the described implementations are merely some implementations of the present disclosure, rather than all the implementations.

It is considered that the repair device for repairing the valve that has a structure similar to a clip has many defects as mentioned above, the present disclosure provides a novel medical device for repairing a patient's valve. The medical device provided by the present disclosure includes a pad configured to be located among a plurality of leaflets of the valve, to make the valve periodically open and close by cooperating with the plurality of leaflets.

By the pad located among the plurality of leaflets, the medical device provided by the present disclosure is able to improve the closure condition of the valve, so that the valve is properly closed, and thus the regurgitation of blood is effectively prevented or reduced. Furthermore, the medical device provided by the present disclosure has less interference to blood flow, and therefore is able to reduce the possibility of stenosis. In addition, the medical device provided by the present disclosure has a minimal influence on the movement of the leaflets, so that fewer adverse effects on the structure and function of the leaflets are caused.

The valve involved in the present disclosure may be heart valves. In particular, the valve may be a mitral valve or tricuspid valve. When the valve is a mitral valve, the plurality of leaflets involved in the present disclosure may be the two leaflets of the mitral valve. When the valve is a tricuspid valve, the plurality of leaflets may be the three leaflets of the tricuspid valve. It should be understood that, the medical device provided by the present disclosure is not limited to application in mitral and tricuspid valves, but may also be applied to other valves.

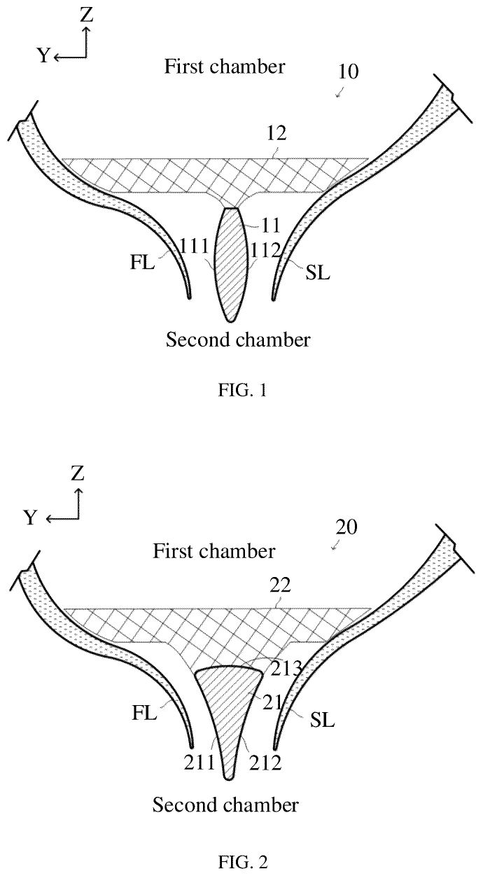

The medical device provided by the present disclosure will be described below with reference to the drawings. It should be noted that, in FIG. 1 to FIG. 5 of the present disclosure, an arrow X is used for indicating a width direction of a pad, an arrow Y is used for indicating a thickness direction of the pad, and an arrow Z is used for indicating a height direction of the pad. FIG. 1 is a schematic structural diagram of a medical device 10 according to an embodiment of the present disclosure, and FIG. 1 illustrates a state that the medical device 10 is placed in a heart valve of a patient.

In FIG. 1 (and other figures of the present disclosure), the heart valve is located between a first chamber and a second chamber of the heart, to allow blood to flow from the first chamber to the second chamber while preventing flowing in a reverse direction. The heart valve includes a pair of leaflets FL, SL matching with each other.

In a normal condition, the leaflets FL, SL are able to naturally coaptate to close the valve and separate to open the valve during a cardiac cycle, so that the blood is allowed to flow from the first chamber to the second chamber while preventing flowing in a reverse direction. However, for a patient with valvular insufficiency, when the valve is required to close, the leaflets FL, SL fail to achieve proper coaptation. For example, a degree of coaptation is significantly reduced, and even there is a gap between the leaflets FL, SL. Therefore, this makes the blood flow from the second chamber back into the first chamber through the valve, and thus resulting regurgitation during systole.

The valve shown in FIG. 1 (as well as in other figures of the present disclosure) may be a mitral valve. Correspondingly, the first chamber is the left atrium, the second chamber is the left ventricle, and one of the leaflets FL, SL is the anterior leaflet while the other is the posterior leaflet. It should be understood that, the medical device provided in the present disclosure is not limited to application in the mitral valve, but may also be applied to other heart valves.

Referring to FIG. 1, the medical device 10 includes a pad 11. When the medical device 10 is implanted in a patient's heart, the pad 11 is located between the leaflet FL and leaflet SL. The pad 11 has a pair of coaptation surfaces 111, 112. When the medical device 10 is implanted in the patient's heart, the pair of coaptation surfaces 111, 112 respectively face the pair of leaflets FL, SL, namely the coaptation surface 111 faces the leaflet FL and the coaptation surface 112 faces the leaflet SL. With the changes of the cardiac cycle, each coaptation surface of the pair of coaptation surfaces 111, 112 periodically coaptates and separates from the leaflet it faces. Specifically, when the valve closes, the coaptation surface 111 coaptates the leaflet FL and the coaptation surface 112 coaptates the leaflet SL to prevent the blood flowing from the second chamber back to the first chamber. When the valve opens, the leaflets FL, SL separate from coaptation surfaces 111, 112 respectively to allow the blood to flow from the first chamber into the second chamber. In an example, a coaptation length between each coaptation surface and its corresponding leaflet may range from 6 mm to 12 mm. In an example, the coaptation surfaces 111, 112 may be smooth transition curved surfaces to reduce resistance to the blood flow.

In this implementation, the valve is able to be opened and closed periodically by their own movement, and effective closure of the leaflets is increased through the coaptation of the pad and the leaflet during systole. Therefore, it not only able to allow the blood to automatically flow from the first chamber to the second chamber during diastole, but also reverse flow during systole caused by poor coaptation of the original two leaflets is effectively prevented.

Referring again to FIG. 1, the medical device 10 may further include a support member 12. When the medical device 10 is implanted in a patient's heart, the support member 12 is located in the first chamber and is connected to the pad 11, so that the pad 11 is positioned between the pair of leaflets SL, FL. By placing the support member 12 in the first chamber, the pad 11 is able to be reliably positioned between the pair of leaflets FL, SL. In an example, the pad 11 may be rigidly connected to the support member 12 to consistently maintain the pad 11 in a proper position, avoiding changes in the position of the pad 11 due to the influence of blood flow or the leaflets FL, SL. If the pad 11 cannot be maintained in a proper position, it cannot be ensured that the pair of leaflets FL, SL will achieve good coaptation with the pair of coaptation surfaces 111, 112 each time the valve closes.

The support member may be implemented in various ways, which are not specifically limited in the present disclosure.

As an example, referring again to FIG. 1, the support member 12 may be annular in shape. When the medical device 10 is implanted in a patient's heart, the support member 12 may be placed at the annulus of the valve of the heart. In some embodiments, the support member 12 may have a mesh frame structure. During delivery, the support member 12 may firstly be folded to reduce its volume, to make the delivery process be easy. Once the support member 12 is delivered to a target position, it may be unfolded again.

It should be understood that, the implementation of the support member is not limited to the above, as long as it is able to position the pad between the pair of leaflets of the valve. For example, in some embodiments, the support member may also be semi-annular or partially annular. In some embodiments, the support member may be made of a flexible material, so that it is able to deform in accordance with systole and diastole of the first chamber, and thereby reducing adverse effects on physiological functions of the heart.

FIG. 2 is a schematic structural diagram of a medical device 20 according to another embodiment of the present disclosure, showing a state that the medical device 20 is placed at a heart valve of a patient.

Referring to FIG. 2, the medical device 20 includes a pad 21. In some embodiments, the pad 21 may be configured such that its thickness gradually increases toward a first chamber (that is, along the height direction, the pad has a gradually increasing cross-sectional area from its bottom end to its top end), thereby better matching with shapes of a pair of leaflets FL, SL, and thus the closure of the valve is more effectively achieved.

When the medical device 20 is implanted in the patient's heart, the pad 21 is located between the pair of leaflets FL, SL. The pad 21 has a pair of coaptation surfaces 211, 212. When the medical device 20 is implanted in the patient's heart, the pair of coaptation surfaces 211, 212 respectively face the pair of leaflets FL, SL. As the pair of leaflets FL, SL move, each coaptation surface periodically coaptates and separates from the leaflet it faces.

The pair of coaptation surfaces 211, 212 may both be inwardly concave surfaces, that is, concave surfaces, and the concave surfaces are recessed toward the interior of the pad 21. Specifically, when the medical device 20 is implanted in the patient's heart, the coaptation surface 211 may be recessed in a direction away from the leaflet it faces (that is, the leaflet FL), and the coaptation surface 212 may be recessed in a direction away from the leaflet it faces (that is, the leaflet SL).

In this way, the pair of coaptation surfaces are able to better match with the leaflets they face respectively, so that the pair of coaptation surfaces better coaptate the leaflets they face respectively.

As an example, referring again to FIG. 2, when the medical device 20 is implanted in the patient's heart, the coaptation surface 211 may be constructed to gradually extend toward a side where the leaflet FL it faces is located (that is, the left side in FIG. 2) in a direction trending towards the first chamber. That is, along a height direction of the pad 21, from the bottom end to the top end of the pad 21, included angles between tangent lines of points on the coaptation surface 211 on the same longitudinal section and the height direction gradually increase. Similarly, the coaptation surface 212 may be constructed to gradually extend toward a side where the leaflet SL it faces is located (that is., the right side in FIG. 2) in a direction trending towards the first chamber. That is, along the height direction of the pad 21, from the bottom end to the top end of the pad 21, included angles between tangent lines of points on the coaptation surface 212 on the same longitudinal section and the height direction gradually increase. The pair of coaptation surfaces 211, 212 are smooth curved surfaces respectively. The pad 21 also includes a top surface 213 and a bottom surface, an end of the top surface 213 connected to the pair of coaptation surfaces 211, 212 is the top end, and an end of the bottom surface connected to the pair of coaptation surfaces 211, 212 is the bottom end.

In some cases, there may still be a small amount of blood flowing from the second chamber back into the first chamber. By constructing the pair of coaptation surfaces to be concave in directions away from the leaflets they face respectively, and to gradually extend toward the sides where the leaflets they face are located in the direction trending towards the first chamber respectively, so that the flow direction of blood regurgitating from the second chamber to the first chamber is able to be changed, thereby the blood flow being guided to one side to prevent it from directly impacting the first chamber, and thus the direct “impact” of the regurgitation on the patient's atrium and pulmonary veins is reduced to reduce adverse effects on the cardiac function.

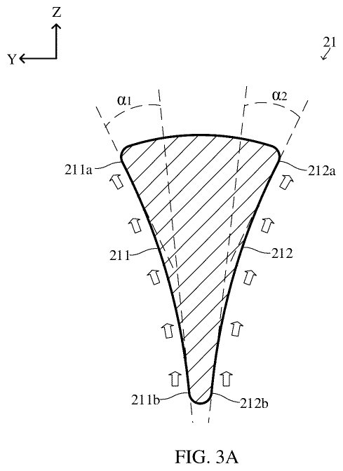

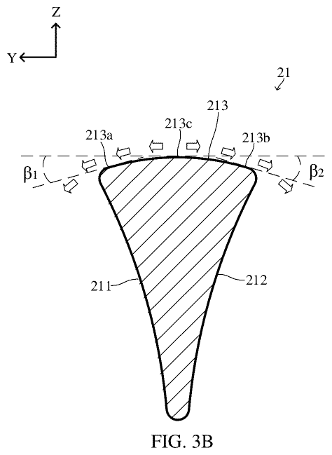

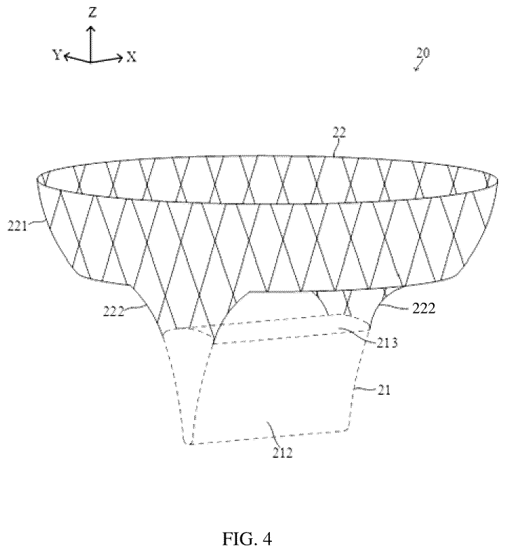

FIG. 3A is a schematic structural diagram of the pad 21. In FIG. 3A, thick arrows are used for schematically indicating the changing trend of the flow direction of the blood flow that flows from the second chamber to the first chamber under the guidance of the pair of coaptation surfaces 211, 212.

Referring to FIG. 2 and FIG. 3A, the coaptation surface 211 has a first edge portion 211a and a second edge portion 211b located at two opposite ends (a top end and a bottom end) thereof, respectively. When the medical device 20 is implanted in the patient's heart, the first edge portion 21la is closer to the first chamber than to the second edge portion 211b. Included angles between tangent planes at positions on the coaptation surface 211 and a tangent plane at the second edge portion 211b gradually increase as the positions on the coaptation surface 211 trends towards the first edge portion 211a. An included angle (acute angle) α1 formed between a tangent plane at the first edge portion 21la and the tangent plane at the second edge portion 211b may range from greater than or equal to 10° to less than or equal to 45°. Preferably, the range of al may be from greater than or equal to 15° to less than or equal to 30°.

The coaptation surface 212 has a first edge portion 212a and a second edge portion 212b located at two opposite ends thereof, respectively. When the medical device 20 is implanted in the patient's heart, the first edge portion 212a is closer to the first chamber than to the second edge portion 212b. Included angles between tangent planes at positions on the coaptation surface 212 and a tangent plane at the second edge portion 212b gradually increase as the positions on the coaptation surface 212 approach the first edge portion 212a. That is, on the same longitudinal section, included angles between tangent lines of points on the coaptation surface 212 and the tangent line of the second edge portion 212b gradually increase as the points on the coaptation surface 212 trends towards the first edge portion 212a. An included angle (acute angle) α2 formed between a tangent plane at the first edge portion 212a and the tangent plane at the second edge portion 212b ranges from greater than 45° to less than 90°. The included angle al and the included angle α2 may be equal or unequal, which is not specifically limited in the present disclosure.

The greater the included angle between the tangent plane at the first edge portion and the tangent plane at the second edge portion, the greater the change in the flow direction of blood flow that flows from the second chamber to the first chamber caused by the coaptation surface. By setting the included angle between the two within the range of 45° to 90°, the blood flow that flows from the second chamber to the first chamber is effectively guided to one side while ensuring proper coaptation between the leaflets and the coaptation surfaces respectively.

It may be understood that, in other embodiments, if the device is used for a valve with three leaflets, the pad may have a third coaptation surface. At least a portion of the third coaptation surface periodically coaptates and separates from the it faces. Meanwhile, the third coaptation surface may also have any of the configurations of the first or second coaptation surfaces described in the above embodiments and achieve the same function.

Referring again to FIG. 2, the pad 21 may also include a top surface 213 and a bottom surface, the top surface 213 and the bottom surface may be outwardly convex surfaces, that is, convex surfaces, and the convex surface protrudes toward the exterior of the pad 21. Specifically, when the medical device 20 is implanted in the patient's heart, the top surface 213 of the pad 21 is located at an end of the pad 21 close to the first chamber, and the top surface 213 faces the first chamber and protrudes toward the first chamber. The bottom surface of the pad 21 is located at an end of the pad close to the second chamber, and the bottom surface faces the second chamber and protrudes toward the second chamber.

To better match with the pair of leaflets of the valve, the thickness of the pad may be constructed to gradually increase towards the first chamber, which may cause the blood to be more easily accumulated at the top of the pad. In the above implementation of the present disclosure, since the pad has a top surface that is convex (in the direction toward the first chamber), so that the accumulation of the blood at the top of the pad is effectively prevented or reduced.

In an example, referring again to FIG. 2, when the medical device 20 is implanted in the patient's heart, the top surface 213 of the pad 21 may be constructed to trend towards the side where each of the pair of leaflets FL, SL is located while gradually extend toward the second chamber from its middle portion.

Specifically, as shown in FIG. 2, the top surface 213 trends towards the side where the leaflet FL is located while (that is, the left side in FIG. 2) gradually extending toward the second chamber or away from the support member 22 (that is, gradually extending downward in FIG. 2) from its middle portion. Meanwhile, the top surface 213 approaches the side where the leaflet SL is located (that is, the right side in FIG. 2) while gradually extending toward the second chamber or away from the support member 22 (that is, gradually extending downward in FIG. 2) from its middle portion. In other words, the top surface 213 has a configuration that is higher in the middle and lower at both ends

FIG. 3B is a schematic structural diagram of the pad 21. In FIG. 3B, thick arrows are used for schematically indicating the changing trend of the flow direction of blood flow that flows from the first chamber to the second chamber under the guidance of the top surface 213 of the pad 21.

Referring to FIG. 2 and FIG. 3B, the top surface 213 has edge portions 213a, 213b at two ends thereof in the thickness direction of the pad 21, respectively. When the medical device 10 is implanted in the patient's heart, the edge portion 213a is closer to the leaflet FL (or closer to the coaptation surface 211) than the edge portion 213b, and the edge portion 213b is closer to the leaflet SL (or closer to the coaptation surface 212) than the edge portion 213a.

An included angle between a tangent plane at the middle portion 213c of the top surface 213 and a tangent plane at the edge portion 213a is defined as β1, and an included angle between the tangent plane at the middle portion 213c and a tangent plane at the edge portion 213b is defined as β2. The included angles β1 and β2 may range from greater than or equal to 10° to less than or equal to 45°. In an example, the included angles β1 and β2 may range from greater than or equal to 15° to less than or equal to 30°. In an example, the top surface 213 may be a smooth transition curved surface to reduce resistance to blood flow.

The configuration of the top surface in FIG. 2 and FIG. 3B is able to better guide the blood flow that flows from the first chamber to the second chamber, so that obstruction to the blood flow that flows from the first chamber to the second chamber is reduced.

FIG. 4 is an axonometric schematic structural diagram of the medical device 20.

As shown in FIG. 4, the support member 22 includes a main body portion 221 and a pair of connecting portions 222. As an example, the main body portion 221 may be annular in shape, and is constructed such that is adapted to be mounted at the valve annulus. Certainly, in other examples, the main body portion 221 may be semi-annular or other shapes. The pair of connecting portions 222 extend respectively to two sides of the pad 21 in the width direction and are connected to the pad 21.

The pair of leaflets are located on two sides of the pad in the thickness direction, and the first chamber and the second chamber are located on two sides of the pad in the height direction. As an example, the thickness direction, height direction and width direction of the pad are mutually perpendicular to each other. When the valve opens, most of the blood will flow along the height direction and passes through two sides of the pad in the thickness direction, and eventually flows into the second chamber. Since most of the blood flows through the two sides of the pad in the thickness direction, so that this configuration of the support member is able to reduce the obstruction to the blood flow caused by the portions of the support member connected to the pad to a greater extent, and thus the influence of the medical device on the patient's normal physiological functions is reduced.

It should be noted that, in the various embodiments of the present disclosure described above, any one of the leaflet FL and leaflet SL may be the “first leaflet” mentioned in other sections (for example, the Summary section). Correspondingly, the coaptation surface facing the “first leaflet” may be the “first coaptation surface”. The other one of the leaflet FL and leaflet SL may be the “second leaflet” mentioned in other sections (for example, the Summary section). Correspondingly, the coaptation surface facing the “second leaflet” may be the “second coaptation surface”.

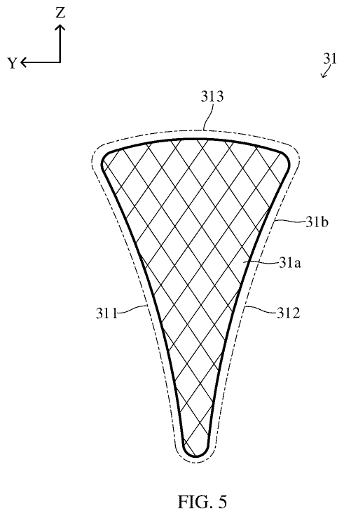

FIG. 5 is a schematic structural diagram of a pad 31 of a medical device according to an embodiment of the present disclosure.

As shown in FIG. 5, the pad 31 includes an expandable mesh frame 31a and a cover 31b covering the mesh frame 31a. The pair of coaptation surfaces 311, 312 and the top surface 313 may be a portion of an outer surface of the cover 31b. In an example, the cover 31b may be made of a biocompatible material and has a smooth outer surface to avoid or reduce the wear of the leaflets or discomfort when matching with the leaflets. In another implementation, the cover 31b may be provided merely on the top end of the pad without covering on the coaptation surface. In this way, endothelialization of the coaptation surface is developed more quickly, so that the influence on the leaflet tissue when the leaflets come into contact with the coaptation surface is reduced.

The shape and size of the pad mainly depend on the mesh frame 31a. During delivery, the mesh frame 31a may be folded to reduce an overall size of the pad 31, thereby facilitating the delivery process. After the pad 31 is delivered to a target position, the mesh frame 31a may be unfolded to make the pad 31 have an appropriate size and shape. Additionally, this configuration having the mesh frame 31a allows a doctor to adaptively adjust the shape and size of the pad 31 according to differences in different patients' valve, so that the pad 31 is able to better fit with the patient's valves. The cover 31b covering the outer side of the mesh frame 3 la provides the pad 31 with a smooth surface, which on one hand helps to prevent or reduce tissue overgrowth or blood adhesion on the pad 31, and on the other hand enables the pad 31 to coaptate the leaflets tightly.

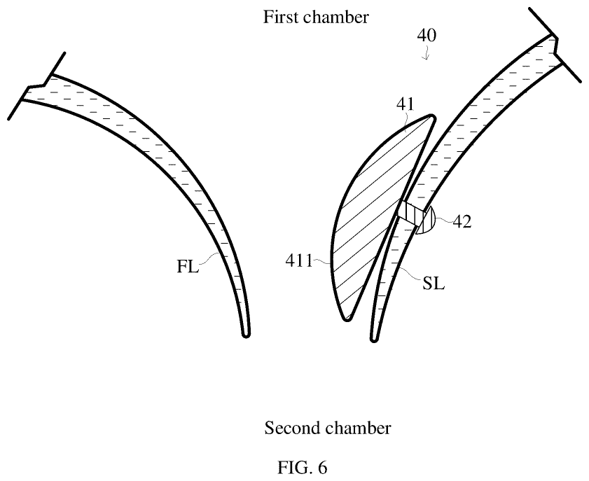

FIG. 6 is a schematic structural diagram of a medical device 40 according to an embodiment of the present disclosure, showing a state that the medical device 40 is placed at a patient's heart valves.

As shown in FIG. 6, the medical device 40 includes a pad 41 having a first coaptation surface 411. When the medical device 40 is implanted in the patient's heart, the pad 41 is located between a first leaflet (FL) and a second leaflet (SL) of the heart valves and moves with the second leaflet SL. The first coaptation surface 411 faces the first leaflet FL and periodically coaptates and separates from the first leaflet FL as the pad 41 moves with the second leaflet SL. In an example, when the first coaptation surface 411 coaptates with the first leaflet FL, a coaptation length between them may range from 6 mm to 12 mm.

Through the pad 41 locating between the pair of leaflets, the medical device 40 is able to improve the closure of the valve, so that the first leaflet FL coaptate the second leaflet SL properly, and thus the regurgitation of the blood is effectively prevented or reduced. Additionally, the medical device 40 has less interferes with blood flow, so that the possibility of stenosis is reduced. Furthermore, the medical device 40 does not affect or has less influence on the motion of the leaflet, so that no or less problems is caused to the function and structure of the leaflets. In other words, by using the medical device 40 provided by the present disclosure, there is no restriction or obstruction on the natural motion of the valve (closing, opening). Meanwhile, it is not associated with the valve's supporting structures, so that there are no adverse effects to be caused. That is, the physiological cyclic motion of each leaflet will not be disturbed or be less disturbed. As a result, the medical device 40 will cause less fewer problems to the structure and function of the leaflets. Moreover, the valve is able to cyclically open and close through their own natural motion, and during systole, sufficient closure of the leaflets is increased through the coaptation between the pad 41 and the leaflet. This not only allows the blood to flow from the first chamber to the second chamber during diastole, but also effectively prevents the regurgitation during systole that caused by the insufficient coaptation between the two leaflets.

The pad 41 may be attached to the second leaflet SL to move with the second leaflet SL. In other words, the pad 41 may be attached to the second leaflet SL in such a way that it follows the motion of the second leaflet SL and becomes a portion of the second leaflet SL, so that the first coaptation surface 411 is able to periodically coaptate and separate from the first leaflet FL with the movement of the second leaflet SL.

As an exemplary implementation, referring again to FIG. 6, the medical device 40 may also include a fixing member 42. In an example, the fixing member 42 may be a pin 42. When the medical device 40 is implanted in the patient's heart, the pin 42 may penetrate through the second leaflet SL to attach the pad 41 to the second leaflet SL, so that the pad 41 is able to move with the second leaflet SL.

The pin 42 may be integrally formed with the pad 41, or the two may be separate members, which is not specifically limited in the embodiments of the present disclosure.

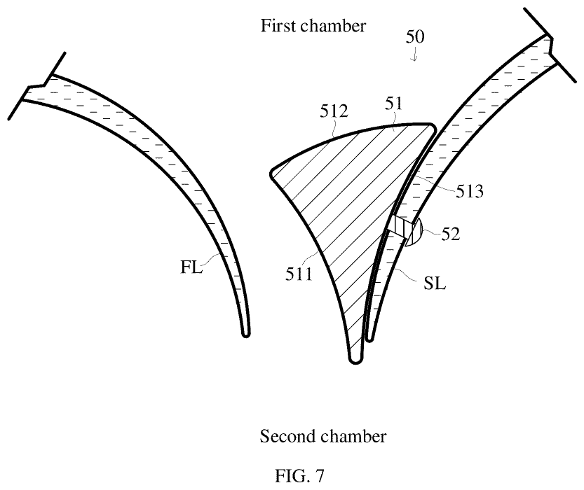

FIG. 7 is a schematic structural diagram of a medical device 50 according to still another embodiment of the present disclosure, showing a state that the medical device 50 is placed at a patient's heart valves.

As shown in FIG. 7, the medical device 50 includes a pad 51 having a first coaptation surface 511. In some embodiments, the pad 51 may be configured such that its thickness gradually increases towards the first chamber (that is, along the height direction, the pad has a gradually increasing larger cross-sectional area from the bottom end to the top end of the pad), so that the pad 51 better matches with the shapes of the first leaflet FL and the second leaflet SL, and thus the closure of the valve is improved.

When the medical device 50 is implanted in the patient's heart, the pad 51 is located between the first leaflet FL and the second leaflet SL and moves with the second leaflet SL. The first coaptation surface 511 faces the first leaflet FL and periodically coaptates and separates from the first leaflet FL as the pad 51 moves with the second leaflet SL.

The first coaptation surface 511 may be a recessed surface. More specifically, when the medical device 50 is implanted in the patient's heart, the first coaptation surface 511 may be recessed in a direction away from the first leaflet FL. This configuration allows the first coaptation surface 511 to better match with the first leaflet FL, so that better coaptation between the first coaptation surface 511 and the first leaflet FL is achieved.

Referring again to FIG. 7, in a non-limiting example, when the medical device 50 is implanted in the patient's heart, the first coaptation surface 511 gradually extends toward the side where the first leaflet FL is located (that is, the left side in FIG. 7) in the direction trending towards the first chamber.

In some cases, there may still be a small amount of blood flow from the second chamber back into the first chamber. The first coaptation surface 511 (away from the first leaflet FL) is concave and gradually extends toward the side where the first leaflet FL is located in the direction trending towards the first chamber, this configuration enables to increase the resistance of blood flow, so that the regurgitant is reduced, and the flow direction of the blood flow that flows from the second chamber to the first chamber is changed. Therefore, the blood flow is guided to one side, so that the blood flow is prevented from directly impacting the first chamber, and thus the adverse effects on the patient's cardiac function caused by the regurgitant that flows from the second chamber to the first chamber is reduced.

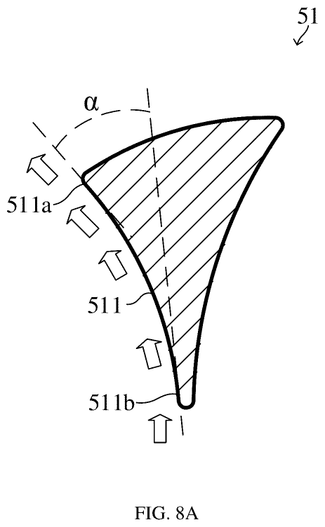

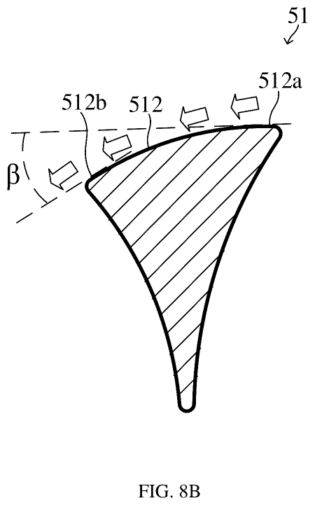

FIG. 8A is a schematic structural diagram of a pad 51. As shown in FIG. 8A, the direction indicated by the thick arrow may represent the flow direction of blood that flows from the second chamber to the first chamber under the guidance of the first coaptation surface 511.

As an exemplary implementation, referring to FIG. 7, FIG. 8A, and FIG. 8B, the first coaptation surface 511 has a first edge portion 511a and a second edge portion 511b located at two opposite ends (the top end and the bottom end) thereof along the height direction, respectively. When the medical device 50 is implanted in a patient's heart, the first edge portion 511a is closer to the first chamber than the second edge portion 511b. The pad 51 also includes a top surface 512 and a bottom surface, an end where the top surface 512 is connected to the first coaptation surface 511 is the top end, and the end where the bottom surface is connected to the first coaptation surface 511 is the bottom end.

Included angles between tangent planes at positions on the first coaptation surface 511 and a tangent plane at the second edge portion 511b gradually increase as they approach the first edge portion 511a. An included angle (acute angle) α formed between a tangent plane at the first edge portion 511a and the tangent plane at the second edge portion 511b may range from greater than or equal to 10° to less than or equal to 45°. In an example, the included angle a may range from greater than or equal to 15° to less than or equal to 30°.

The greater the included angle a between the tangent plane at the first edge portion 511a and the tangent plane at the second edge portion 511b, the greater the flow direction of blood flow that flows from the second chamber to the first chamber changed by the first coaptation surface 511. The included angle a of the two is set within this range, so that the blood flow that flows from the second chamber to the first chamber is able to be effectively guided to one side on the basis of ensuring the good coaptation of the first leaflet FL and the first coaptation surface 511.

In an example, referring again to FIG. 7, the pad 51 may also include a top surface 512, and the top surface 512 may be a convex surface. When the medical device 50 is implanted in the patient's heart, the top surface 512 of the pad 51 is located at the end of the pad closer to the first chamber, and the top surface 512 faces the first chamber and protrudes (toward the first chamber), that is, the top surface 512 is a convex surface, and this convex surface protrudes toward the outside of the pad 51.

To better match with the first leaflet FL and the second leaflet SL, the thickness of the pad 51 gradually increases towards the first chamber, which may lead to the accumulation of the blood at the top of the pad 51. Since the pad 51 has a top surface 512 that protrudes (toward the first chamber), so that the accumulation of the blood at the top of the pad 51 is effectively prevented or reduced.

In a non-limiting example, referring again to FIG. 7, when the medical device 50 is implanted in the patient's heart, the top surface 512 of the pad 51 approaches the second chamber while gradually extending toward the side where the first leaflet FL is located (that is, the left side in FIG. 7). In other words, along the height direction of the pad 51, from the bottom end to the top end of the pad 51, the cross-sectional area of the pad 51 increases.

This configuration of the top surface 512 of the pad 51 is able to effectively prevent or reduce the accumulation of the blood at the top of the pad 51. Additionally, this configuration of the top surface 512 of the pad 51 is also able to reduce obstruction and interference to blood flow that flows from the first chamber to the second chamber caused by the pad 51, and thereby better guiding blood flow to flow from the first chamber into the second chamber.

FIG. 8B is a schematic structural diagram of the pad 51. As shown in FIG. 8B, the direction indicated by the thick arrow may represent the flow direction of blood flow that flows from the first chamber to the second chamber under the guidance of the top surface 512 of the pad 51.

In an example, referring to FIGS. 7 and 8B, the top surface 512 of the pad 51 includes a third edge portion 512a and a fourth edge portion 512b located at two opposite ends thereof in the thickness direction, respectively. When the medical device 50 is implanted in the patient's heart, the fourth edge portion 512b is closer to the second chamber than the third edge portion 512a.

Included angles between tangent planes of the top surface 512 and a tangent plane at the third edge portion 512a gradually increase as they approach the fourth edge portion 512b. An included angle β formed between the tangent plane at the third edge portion 512a and a tangent plane at the fourth edge portion 512b may range from greater than or equal to 10° to less than or equal to 45°. In an example, the included angle β may range from greater than or equal to 15° to less than or equal to 30°.

In this manner, the top surface 512 of the pad 51 is able to better guide the blood to flow from the first chamber to the second chamber, so that the obstruction caused by the pad 51 to the blood flow is reduced.

In a specific example, referring again to FIG. 7, the pad 51 may also include a second coaptation surface 513. The second coaptation surface 513 may be a recessed surface, that is, the second coaptation surface 513 may be a concave surface, this concave surface is recessed toward the interior of the pad 51. More specifically, when the medical device 50 is implanted in the patient' s heart, the second coaptation surface 513 faces the second leaflet SL and is recessed away from the second leaflet SL. Additionally, when the medical device 50 is implanted, the second coaptation surface 513 approaches the first chamber while gradually extending toward the side where the second leaflet SL is located (that is, the right side in FIG. 7). As the pad 51 moves with the second leaflet SL, the second coaptation surface 513 coaptates the second leaflet SL.

This configuration enables the second coaptation surface 513 of the pad 51 to better match with and fit with the second leaflet SL, so that the accumulation of the blood between the second coaptation surface 513 and the second leaflet SL is effectively prevented or reduced.

In a non-limiting example, referring again to FIG. 7, the medical device 50 may also include a pin 52. When the medical device 50 is implanted in the patient's heart, the pin 52 may penetrate through the second leaflet SL to attach the pad 51 to the second leaflet SL to make the pad 51 move with the second leaflet SL.

The pin 52 may be integrally formed with the pad 51, or the two may be separate members, which is not specifically limited in the embodiments of the present disclosure.

There are various methods for attaching the pad to the second leaflet SL, which are not specifically limited in the present disclosure. One possible implementation is exemplarily described with reference to the accompanying drawings below.

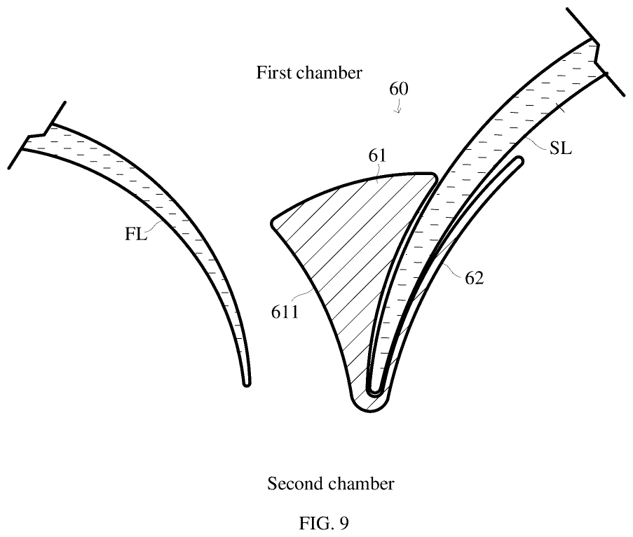

FIG. 9 is a schematic structural diagram of a medical device 60 according to further another embodiment of the present disclosure. FIG. 9 illustrates a state that the medical device 60 is placed at heart valves of a patient. The medical device 60 is substantially the same as the medical device 20, and for the sake of brevity, the similarities will not be described herein again.

Referring to FIG. 9, the medical device 60 includes a pad 61 having a first coaptation surface 611. When the medical device 60 is implanted in the patient's heart, the pad 61 is located between the first leaflet FL and the second leaflet SL and moves with the second leaflet SL. As the second leaflet SL moves, the first coaptation surface 611 periodically coaptates and separates from the first leaflet FL, so that the valve is cyclically closed and opened.

The medical device 60 also includes a clamping member 62, and the clamping member 62 may be integrally formed with the pad 61. Certainly, in other embodiments, the clamping member 62 may also be a member that is separate from the pad 61. When the medical device 60 is implanted in the patient's heart, the clamping member 62 clamps the second leaflet SL to attach the pad 61 to the second leaflet SL, thereby enabling the pad 61 to move with the second leaflet SL

The present disclosure is not limited to attaching the pad to the second leaflet SL. Other implementations may also be used for positioning the pad between the first leaflet FL and the second leaflet SL and enabling the pad to move with the second leaflet SL.

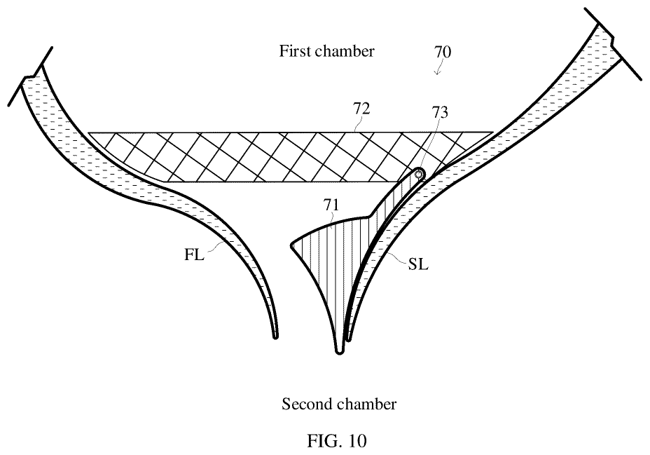

FIG. 10 is a schematic structural diagram of a medical device 70 according to yet another embodiment of the present disclosure. FIG. 10 illustrates a state that the medical device 70 is placed at heart valves of a patient. The medical device 70 is substantially to the same as the medical device 20, and for the sake of brevity, the similarities will not be described herein again.

Referring to FIG. 10, the medical device 70 includes a pad 71 and a support member 72. When the medical device 70 is implanted in the patient's heart, the support member 72 is located in the first chamber, and the pad 71 is connected to the support member 72 in a manner enabling it to move with the second leaflet SL, so that the pad 71 is positioned between the first leaflet FL and the second leaflet SL while following the motion of the second leaflet SL.

In a specific implementation, the pad 71 may be hinged with the support member 72 to make the pad 71 move with the second leaflet SL. In an example, as shown in FIG. 10, the medical device 70 may also include a pivot shaft 73, and the pad 71 is hinged with the support member 72 through the pivot shaft 73. For instance, the pivot shaft 73 may be fixed to the pad 71 and is pivotally connected to the support member 72, and thereby achieving the hinged connection between the pad 71 and the support member 72. Alternatively, the pivot shaft 73 may be fixed to the support member 72 and pivotally connected to the pad 71, and thereby achieving the hinged connection between the pad 71 and the support member 72.

In another implementation, the pad 71 may be connected to the support member via a flexible member (or portion) to enable the pad 71 to move with the second leaflet SL.

By the support member 72 placed in the first chamber, the pad 71 is reliably positioned between the first leaflet FL and the second leaflet SL. The pad 71 is connected to the support member 72 in a manner that the pad 71 is allowed to move with the second leaflet SL, so that as the second leaflet SL moves, the pad 71 moves with the second leaflet SL, and thus the first coaptation surface coaptates and separates from the first leaflet FL periodically.

In an embodiment, the support member 72 may be annular to reduce the influence on the blood flow. Certainly, in other embodiments, the support member 72 maybe semi-annular or partially annular. In an implementation, the support member 72 may be constructed to be relatively soft, or more specifically have flexibility to adaptively deform with the systole and diastole of the first cardiac chamber, so that the adverse effects on the cardiac function are reduced.

There are various implementations of the support member 72, which is not specifically limited in the present disclosure. In a non-limiting example, referring again to FIG. 10, the support member 72 may be in a mesh frame shape that is able to be folded and unfolded. During implanting, the support member 72 may first be folded and then unfolded in the first chamber.

In some embodiments, the support member 72 may be placed at the valve annulus to enable the support member 72 to be reliably fixed.

In related art, a medical device for repairing heart valves includes a pad, and the pad may be delivered between leaflets of the valve of a patient by an operation. When the valve is closed, the pad is able to fill a gap between the leaflets, so that the closure of the valve is improved.

The condition of a patient is continuously developed. After the patient's condition develops to a certain extent, the pad that is initially implanted at the valve may no longer match with the patient's condition. For example, if the condition deteriorates, the pad will no longer be able to adequately fill the gap between the leaflets when the valve closes, so that the regurgitation will reappear. Conversely, if the condition improves (for example, if the ventricle begins to get smaller), the pad may reduce the effective orifice area through which the blood flows across the valve when it open, and thereby causing stenosis.

If the above situations occur, a repeat operation will be required to replace the pad initially implanted in the patient' body with one that matches with the patient's current condition. However, reoperation will increase economic burden on the patient and impair the patient's health.

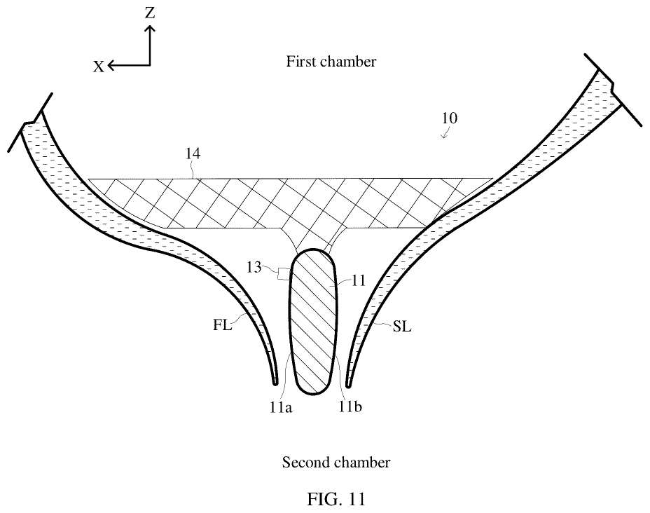

In view of the above problems, the present disclosure provides a medical device. FIG. 11 is a schematic structural diagram of a medical device 10 according to an embodiment of the present disclosure. FIG. 11 illustrates a state that the medical device 10 is placed at heart valves of a patient. The medical device 10 (and other medical devices provided by the present disclosure) provided by the present disclosure is adapted to be implanted in a patient' body to repair the valve of the patient.

In FIG. 11 (and other drawings of the present disclosure), the heart valves are located between a first chamber and a second chamber to allow the blood to flow from the first chamber to the second chamber and preventing flow in the opposite direction. The heart valves include a pair of leaflets FL, SL matching with each other. For ease of description, the leaflet FL will be referred to as the first leaflet, and the leaflet SL will be referred to as the second leaflet hereinafter.

In a healthy heart, the pair of leaflets FL, SL are able to naturally coaptate and separate with the cardiac cycle to periodically close and open the valves, and thereby allowing the blood to flow from the first chamber to the second chamber while preventing flow in the opposite direction. However, for patients with valve insufficiency, when the valve is required to close, the pair of leaflets FL, SL fail to achieve proper coaptation, so that there is a gap between them, thereby the blood may flow through the valve from the second chamber back to the first chamber, and thus resulting in the regurgitation.

It should be noted that, the valve in FIG. 11 (and other drawings of the present disclosure) may be the mitral valve. Correspondingly, the first chamber may be the left atrium, the second chamber may be the left ventricle, and one of the pair of leaflets FL, SL may be the anterior leaflet while the other may be the posterior leaflet. It should be understood that, the medical device provided in the present disclosure is not limited to the application in the mitral valve but may also be applied to other heart valve, such as the tricuspid valve. When the medical device of the present disclosure is applied to the tricuspid valve, it may be located among the three leaflets of the tricuspid valve.

Referring to FIG. 11, the medical device 10 includes a pad 11. When the medical device 10 is implanted in a patient's heart, the pad 11 is located between the pair of leaflets FL, SL. The pad 11 is configured to cooperate with the pair of leaflets FL, SL to enable the valve to periodically open and close. Specifically, when the valve closes, the pad 11 may improve insufficient closure or fill the gap existing between the pair of leaflets FL, SL when closing, and thereby enabling the valve to close properly.

As an example, the pad 11 has a pair of coaptation surfaces 11a, 11b. When the medical device 10 is implanted in the patient's heart, the pair of coaptation surfaces 11a, 11b face the pair of leaflets FL and SL, respectively. For ease of description, the coaptation surface 11a will be referred to as the first coaptation surface, and the coaptation surface 11b will be referred to as the second coaptation surface hereinafter. With the changes of the cardiac cycle, each of the pair of coaptation surfaces 11a, 11b periodically coaptates and separates from the leaflet it faces, and thereby enabling the valve to open and close periodically. For example, a coaptation length between each coaptation surface and its corresponding leaflet may range from 6 mm to 12 mm.

Specifically, when the second chamber is in diastole, the pair of leaflets FL, SL move away from each other to form gaps between the pair of leaflets FL, SL and the pair of coaptation surfaces 11a and 11b, respectively. At this time, the valve opens to allow the blood to flow from the first chamber to the second chamber through these gaps. When the second chamber is in systole, the pair of leaflets FL, SL move toward each other to make the pair of leaflets FL, SL coaptate the pair of coaptation surfaces 11a and 11b, respectively. At this time, the valve closes to prevent the blood flow from the second chamber back to the first chamber.

It should be noted that, in the present disclosure, when the leaflet coaptate a coaptation surface, the leaflet and the coaptation surface may contact and match with each other to prevent the blood from flowing through them.

It should be considered that the patient's condition is continuously developed, the pad may no longer match the patient's condition after being implanted for some time, and may fail to achieve the intended therapeutic effect and even cause adverse reactions. If this situation occurs, another operation would be required to replace the pad which is initially implanted in the patient's body with a pad matching with the patient's current condition. However, undergoing another operation would increase the patient's economic burden and have certain risks, and even impair the patient's health.

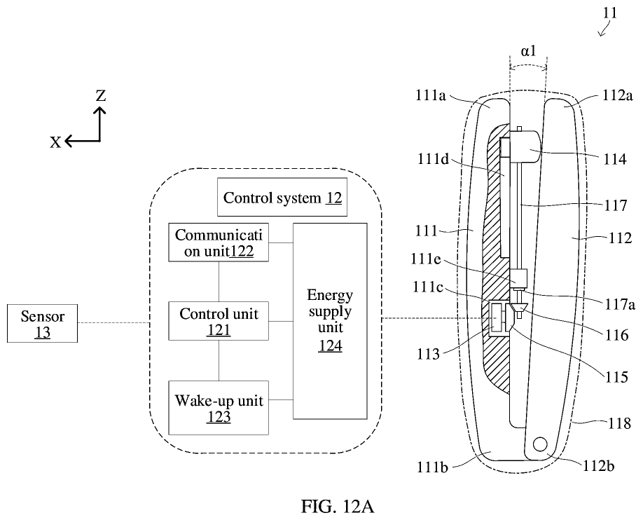

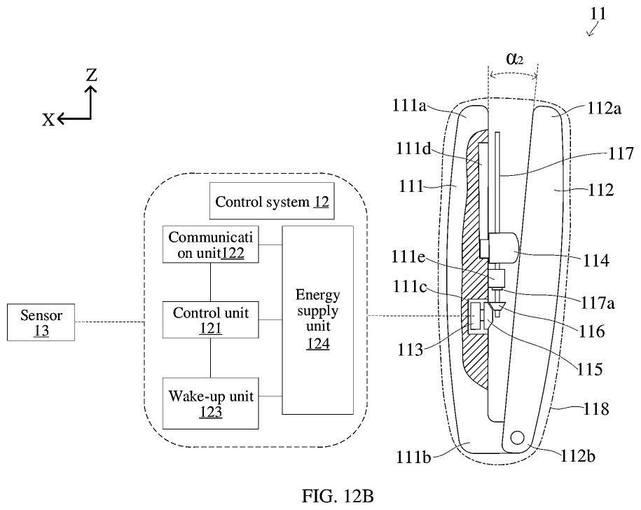

FIG. 12A and FIG. 12B are schematic structural diagrams illustrating the pad 11, a control system 12, a sensor 13, and the like of the medical device 10. As a portion of the medical device 10, the pad 11, the control system 12, and the sensor 13 may all be implanted in the patient's body.

Referring to FIG. 12A and FIG. 12B, the medical device 10 may also include a control system 12, the control system 12 may include a control unit 121, and the pad 11 may include an adjustment mechanism. The control unit 121 is configured to control the adjustment mechanism to enable the adjustment mechanism to adjust a dimension of the pad 11. In an example, the control unit 121 may be configured to control the adjustment mechanism to adjust the thickness of the pad 11. The control unit 121 may include one or more controllers.

In this way, when the patient's condition changes, it is merely necessary to control the adjustment mechanism via the control unit to adjust the dimension of the pad, so that the pad matches with the patient's condition again. Since there is no requirement to perform operation again to replace the pad after the patient's condition develops, the use of this medical device is able to reduce the patient's economic burden and minimize the harm to their health.

In the present disclosure, the dimension of the pad that may be adjusted may be the dimensions of the pad at any dimensionality, but is not limited to its thickness. For example, in some embodiments, the adjustment mechanism may adjust one or more of the thickness, height, or width of the pad under the control of the control unit.

The thickness of the pad may refer to its dimension in the direction from one coaptation surface to the other. In other words, the pair of coaptation surfaces may be located on two sides of the pad along the thickness direction of the pad, respectively. That is, when the pad is implanted at the patient's valves, the pair of leaflets are located on two sides of the pad along the thickness direction of the pad.

The height of the pad may refer to a dimension of the pad in a direction from the first chamber to the second chamber when the pad is installed at the valve. The width of the pad may refer to a dimension of the pad in a direction perpendicular to the height and thickness directions.

For ease of understanding, in the drawings of the present disclosure, an arrow X is used for indicating a thickness direction of the pad, an arrow Y is used for indicating a width direction of the pad, and an arrow Z is used for indicating a height direction of the pad

There are various methods for adjusting the thickness of the pad, which are not specifically limited in the present disclosure.

As an example, referring to FIG. 11 to FIG. 12B, the pad 11 includes a pair of main body members 111, 112. The main body members 111, 112 are stacked in the thickness direction of the pad 11. Each main body member has a free end and a connected end opposite to each other, that is, the main body member 111 has a free end 111a and a connected end 111b, and the main body member 112 has a free end 112a and a connected end 112b. The connected ends 111b, 112b of the main body members 111, 112 are pivotally connected. The adjustment mechanism is configured to adjust an opening degree between the main body members 111, 112 under the control of the control unit 121 to adjust the thickness of the pad 11.

The opening degree between the main body members 111, 112 may be defined by an included angle (acute angle) formed therebetween. In FIG. 12A, the included angle α1 between the main body members 111, 112 is less, so that the opening degree between the main body members 111, 112 is less, and the thickness of the pad 11 is also less. In FIG. 12B, the included angle α₂ between the main body members 111, 112 is greater, so that the opening degree between the main body members 111, 112 is greater, and the thickness of the pad 11 is also greater.

In this manner, the adjustment mechanism is able to regulate the thickness of the pad 11 under the control of the control unit. This implementation has many advantages, such as it is relatively simple to implement, and has a compact structure and high reliability.

It may be understood that, in other examples, the thickness of the pad 11 may also be adjusted in other manners. For instance, in some embodiments, the pad 11 may include a pair of plates stacked in the thickness direction, and the adjustment mechanism may adjust the thickness of the pad by adjusting a space between the plates.

There are various implementations of the adjustment mechanism, which are not specifically limited in the present disclosure.

As an example, the adjustment mechanism may include a driving member 113 and a sliding block 114. The sliding block 114 is slidably disposed between the main body members 111,112 and abuts against the main body members 111,112. The driving member 113 is configured to adjust the opening degree between the main body members 111, 112 by driving the sliding block 114 to slide. Specifically, the adjustment mechanism is configured (under control of the control unit 121) to: increase the opening degree between the main body members 111, 112 by driving the sliding block 114 to move toward the connected ends 111b, 112b of the main body members 111, 112; or decrease the opening degree between the main body members 111, 112 by driving the sliding block 114 to move toward the free ends 111a, 112a of the main body members 111, 112.

In this manner, the adjustment mechanism is able to adjust the opening degree between the pair of main body members 111, 112 under the control of the control unit, and thereby adjusting the thickness of the pad 11. This implementation has many advantages, such as it is relatively simple to implement, and has a compact structure and high reliability.

It may be understood that, in other examples, the adjustment mechanism may be implemented in other manners. For instance, in some embodiments, the adjustment mechanism may include a cam disposed between the pair of main body members, and the driving member may adjust the opening degree between the pair of main body members by driving the cam to rotate.

The sliding block may be driven in various ways, which are not specifically limited in the present disclosure.

As an example, referring to FIG. 12A and FIG. 12B, the driving member 113 may be a motor 113. The adjustment mechanism may also include a pair of meshing bevel gears 115, 116 and a leadscrew 117, and the leadscrew 117 passes through a threaded hole provided in the sliding block 114. The bevel gear 115 is mounted on an output shaft of the electric motor 113, and the bevel gear 116 is mounted on the leadscrew 117.

When the motor 113 rotates under the control of the control unit 121, the driving force is transmitted through the bevel gears 115, 116 to the leadscrew 117 to make the leadscrew 117 rotate. As the leadscrew 117 rotates, it drives the sliding block 114 to slide between the main body members 111, 112, so that the opening degree between the main body members 111, 112 is adjusted, and thus the thickness of the pad 11 is adjusted.

In this manner, the sliding block is driven to slide between the pair of main body members 111, 112, so that the opening degree of the main body members 111, 112 is adjusted, and thus the thickness of the pad 11 is adjusted. This implementation has many advantages, such as it is relatively simple to implement, and has a compact structure and high reliability.

It may be understood that, in other embodiments, the sliding block may also be driven in other manners. For example, in some embodiments, the sliding block may be a magnetic member, and the driving member may be an electromagnetic member, so that the sliding block is driven to slide by an electromagnetic force.

In an example, referring to FIG. 12A and FIG. 12B, the main body member 111 is provided with an accommodating groove 111c, the motor 113 is accommodated in the accommodating groove 111c to avoid occupying an additional space, which is convenient for reducing the overall size of the pad 11. In an example, the main body member 111 also includes an elongated guiding slot 111d, a portion of the sliding block 114 extends into the guiding slot 111d, so that the guiding slot 111d is able to guide the sliding block 114 to slide along a preset trajectory. In an example, the main body member 111 includes a boss 111e, and the boss 111e has a through hole for the leadscrew 117 to pass through to provide support for the leadscrew 117. In an example, the leadscrew 117 is provided with a shoulder 117a, and the shoulder 117a is located on a side of the boss 111e away from the sliding block 114 and abuts against the boss 111e to limit the leadscrew 117.

As a possible implementation, referring to FIG. 12A and FIG. 12B, the pad 11 may include a cover 118, and the cover 118 partially or completely covers the pair of main body members 111, 112. The cover 118 may define an external surface of the pad 11. The pair of coaptation surfaces 11a, 11b of the pad 11 are provided by the cover 118. In other words, the pair of coaptation surfaces 11a, 11b are a portion of the outer surface of the cover 118. In some embodiments, the cover 118 may be made of a material with superior biocompatibility and having a smooth outer surface, and thereby reducing the adverse effect of the pad 11 on the health of the patient.

The control unit 121 may automatically control the adjustment mechanism to adjust the dimension of the pad according to the patient's condition, or may be operated in response to an instruction from a doctor or the patient to control the adjustment mechanism to adjust the dimension of the pad. This is not limited in the present disclosure.

As an example, referring to FIG. 12A and FIG. 12B, the control system 12 may also include a communication unit 122. The communication unit 122 (for example, a communication interface or a signal transceiver) is configured to establish communication connection with a control device (for example, a controller) located outside the patient's body in a wireless manner. For example, the communication unit 122 may, but is not limited to, communicate with the external control device through the following manners: Bluetooth, cellular network, Wi-Fi, electromagnetic field, radio frequency communication, or ultrasonic communication, or the like.

The doctor (or the patient) may operate the control device to send a control instruction to the communication unit 122. The communication unit 122 is configured to receive this control instruction and transmit it to the control unit 121. Upon receiving the control instruction, the control unit 121 is configured to control the adjustment mechanism to adjust the dimensions of the pad 11 based on the control instruction.

After the medical device of the present disclosure is implanted, the patient may perform periodical follow-up examinations to confirm the change of the condition. When it is detected that the pad's dimension no longer matches with the patient's condition, the doctor may send a control instruction to the communication unit of the medical device implanted in the patient's body by the external control device. Upon receiving the control instruction, the control unit may control the adjustment mechanism to appropriately adjust the pad's dimension according to the control instruction, so that the pad matches with the patient's condition again.

As another example, referring to FIG. 12A and FIG. 12B, the medical device 10 may also include a sensor 13, and the senor 13 is in communication connection with the control unit 121. The sensor 13 is configured to sense the patient's physiological information and transmit the sensed physiological information to the control unit 121, so that the control unit 121 controls the adjustment mechanism to adjust the dimension of the pad 11 based on the physiological information.

Illustratively, the physiological information may include, but is not limited to, one or more type of: blood pressure information, blood flow velocity information, blood pH information, blood temperature information, blood oxygen level information, electrocardiographic information, heart sound data, cardiac acceleration information, or cardiac contractility information in the patient's heart. Correspondingly, the sensor 13 in the present disclosure may be configured to sense one or more type of the aforementioned information.

Since there is the sensor 13 for sensing the physiological information that reflects the condition of the patient, the control unit 121 is able to control the adjustment mechanism to adjust the dimension of the pad 11 according to the physiological information, so that the pad 11 is able to always match with the condition of the patient. The medical provided by this implementation not only reduces the need for operation while improving the therapeutic effect, but also reduces the number of follow-up examinations of the patient after implantation, so that the economic burden and time cost of the patient is reduced.

It may be understood that, in some embodiments, the medical device provided by the present disclosure may merely include one of the communication unit 122 and the sensor 13, or may include both the communication unit 122 and the sensor 13, which is not specifically limited in the present disclosure.

There are a plurality of implementations of the sensor 13, which is not specifically limited in the present disclosure.

As an example, referring to FIG. 11, the sensor 13 may include a hemodynamic sensor, for example, a pressure sensor or an accelerometer. The sensor 13 may be attached to the outer surface of the pad 11 to sense physiological information reflecting a blood flow state at the valve. In other words, the physiological information sensed by the sensor 13 is able to reflect the blood flow state at the valve, that is, it is able to reflect whether there is regurgitation or stenosis. Therefore, the control unit 121 is able to adjust the dimension of the pad 11 appropriately based on the physiological information, so that the dimension of the pad 11 better matches with the patient's current condition.

It should be noted that, in other examples, the sensor 13 is attached to the pad 11, but is located in the first chamber or the second chamber to monitor the hemodynamic information of the chamber.

It should be noted that, in other examples, the sensor 13 may not be attached to the pad 11. For instance, in some implementations, the sensor 13 may be mounted on the leaflets. Alternatively, in some embodiments, the sensor 13 may be mounted on other members of the medical device, such as the support member 14.

Furthermore, it should also be noted that, in some embodiments, the medical device provided in the present disclosure may include multiple sensors 13 which may be arranged at different positions-for example, one in the first chamber and another in the second chamber, so that the patient's condition is more accurately determined. Alternatively, a single sensor 13 may has two or more sensing elements to detect hemodynamic information from different chambers or different regions in a same chamber.

In an alternative implementation, the communication unit 122 may be configured to send the physiological information to an external device and receive a control instruction generated by the external device based on the physiological information. The control unit 121 may be configured to control the adjustment mechanism to adjust the dimension of the pad 11 in accordance with the control instruction. In this way, the complexity and power consumption of the medical device 10 is reduced.

To further reduce the energy consumption, in an example (as shown in FIG. 12A and FIG. 12B), the control system 12 may also include a wake-up unit 123 (for example, a controller for waking up). The wake-up unit 123 is configured to wake up one or more of the control unit 121, the communication unit 122 and the sensor 13 based on a preset schedule.

Considering that the patient's condition generally develops slowly, so it is not necessary to frequently adjust the dimension of the pad 11. In this implementation, the control unit 121, the sensor 13, the communication unit 122 and the like may be in a dormant state most of the time. After reaching a preset time point, the wake-up unit 123 may wake up one or more of them. In this way, the energy consumption may be effectively reduced, so that a purpose of power-saving is achieved.

As an example, the preset schedule may include a plurality of cycles, each cycle includes a sleep phase and a wake-up phase. During the sleep phase, all energy-consuming units except the wake-up unit 123 may be in a dormant state to reduce the power consumption. After the wake-up phase begins, the wake-up unit 123 may wake up some or all of the energy-consuming units.

For instance, one sleep phase may last from 6 months to 12 months, and one wake-up phase may last from 3 days to 7 days. The duration and start time of each phase may be set according to a requirement. The doctor may set the preset schedule to match with the follow-up examination plan of the patient, that is, the preset schedule is set so that the medical device 10 is just in the wake-up phase when the patient needs the follow-up examination. In this way, after the doctor checks the patient's condition, a control instruction is sent to the communication unit 122 through an external control device to adjust the dimension of the pad 11 to match with the condition of the patient after the follow-up examination.

In an alternative implementation, the wake-up unit 123 is also configured to wake up the control unit 121 in response to physiological information meeting a preset condition. For example, if the physiological information includes a blood pressure, the preset condition may be that the blood pressure is greater than or less than a preset threshold.

Referring again to FIG. 12A and FIG. 12B, the control system 12 may also include an energy supply unit 124, and the energy supply unit 124 is configured to supply energy to one or more of the control unit 121, the communication unit 122, the wake-up unit 123, and sensor 13.

The energy supply unit 124 may be implemented in various ways, which is not specifically limited in the present disclosure. For instance, in some embodiments, the energy supply unit 124 may be a battery. In other embodiments, the energy supply unit 124 may be an induction coil, so that the doctor or the patient may recharge it via an external device. Alternatively, in some embodiments, the energy supply unit 124 may be an ultrasonic transducer, so that ultrasound waves transmitted by an external device may be converted into electrical energy. In some embodiments, the energy supply unit 124 may also be a device that converts kinetic energy into electrical energy.

To simplify the structure, in some implementations, the control system 12 may be partially or completely located inside the pad 11. Certainly, in some embodiments, the control system 12 may be entirely located outside the pad 11. This is not specifically limited in the present disclosure.

There are various methods for positioning the pad 11 between a pair of leaflets, which are not specifically limited in the present disclosure.

As an exemplary embodiment, with reference to FIG. 11, the medical device 10 may also include a support member 14. When the medical device 10 is implanted in a patient's heart, the support member 14 is located in the first chamber and is connected to the pad 11, so that the pad 11 is positioned between the pair of leaflets (SL, FL). By the support member 14 located in the first chamber, the pad 11 is reliably positioned between the pair of leaflets FL, SL. In an example, the pad 11 may be rigidly connected to the support member 14 to hold the pad 11 in a proper position all the time, so that the position of the pad 11 is prevented from moving due to the influence of the blood flow or the leaflets FL, SL. If the pad 11 cannot be held in the proper position, it cannot be ensured that the pair of leaflets FL, SL will properly coaptate the pair of coaptation surfaces 11a, 11b of the pad 11 respectively each time the valve closes.

The support member 14 may be implemented in various configurations, which are not specifically limited in the present disclosure.

As an example, referring to FIG. 11, the support member 14 may be annular. When the medical device 10 is implanted in the patient's heart, the support member 14 may be placed at the valve annulus of the heart. In some embodiments, the support member 14 may have a mesh frame structure. During delivery, the support member 14 may be folded to reduce its volume. After reaching a target position, the support member 14 may be unfolded.

It may be understood that, the implementation of the support member 14 is not limited to the above examples, as long as it is able to position the pad between the pair of leaflets of the valve. For instance, in some embodiments, the support member 14 may be semi-annular.

The sensor 13 is disposed on the coaptation surface of the pad along the thickness direction of the pad, and in the height direction of the pad, the sensor 13 is configured to be closer to the first chamber than to the second chamber. When the medical device 10 includes the support member 14, the sensor 13 is closer to an end of the pad connected to the support member 14 than an end of the pad away from the support member 14, or the sensor 13 is located at the end of the pad connected to the support member 14. In other embodiments, the medical device 10 may not include the support member 14, and at this time, the sensor 13 may be disposed at an end of the pad closer to the first chamber. For example, the sensor 13 may be placed at the top end of the pad as shown in FIG. 7. When the pair of leaflets match with the pad, the sensor 13 is located in a space formed by the leaflets, the pad and a wall of the left atrium for sensing the pressure of the left atrium. The sensor 13 is disposed at the position which is close to the area where the leaflet coaptates the pad to improve the accuracy and comparability of the sensing data, and meanwhile is not affected by the coaptation of the leaflet and the pad 11 to avoid the accuracy of the sensed data being affected due to the sensor 13 being covered by the leaflet. In an implementation, a side of the sensor 13 is connected to the coaptation surface of the pad 11, and the sensing surface of the sensor faces the first chamber or the support member to further avoid the influence of the leaflet on the sensor and improve the sensing accuracy.

During ventricular systole and diastole, a pressure of the left atrium is able to be directly used for determining the regurgitation and stenosis at the heart valves, so that the adjustment mechanism may adjust the thickness of the pad based on the change of the pressure of the left atrium. For example, during the ventricular systole period, a an abnormally high value of the pressure of the left atrium (for example, greater than a preset value) indicates that there is the regurgitation, at this time, the valve is required to be closed more tightly, so that the thickness of the pad is required to be adjusted by the adjustment mechanism to make the pad become greater. Conversely, during ventricular diastole period, an abnormally high value of the left atrium indicates that there is the stenosis, and in this case, the thickness of the pad is required to be adjusted by adjustment mechanism to make the pad become less, so that the stenosis is reduced or eliminated. If the value of the pressure of the left atrium is not abnormal, the adjustment mechanism does not need to adjust the thickness of the pad. Specifically, when the value of the pressure of the left atrium is abnormally increased, the control unit may notify the doctor this phenomenon and let the doctor to perform corresponding treatment (for example, by using the control unit control the adjustment mechanism to adjust), or it may be automatically adjusted by the control unit.

It should be understood that, although the adjustment mechanism in the above embodiments is used for adjusting the thickness of the pad 11, however, in other embodiments, the adjustment mechanism may be used for adjusting dimensions in other dimensionalities of the pad 11. In the following, an alternative implementation is provided.

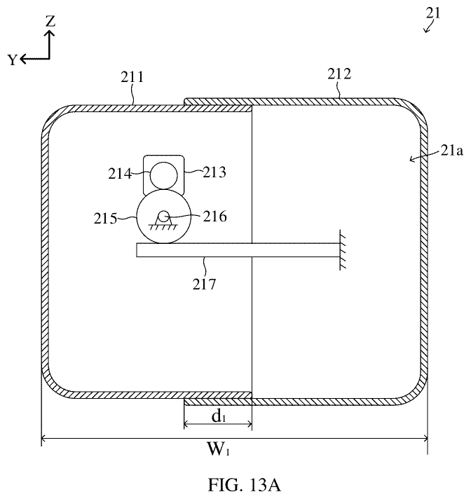

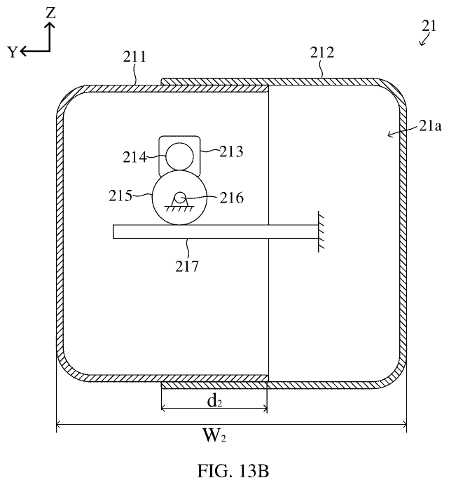

FIG. 13A and FIG. 13B are schematic structural diagrams showing at least a portion of a pad 21 according to another embodiment.

Referring to FIG. 13A and FIG. 13B, the pad 21 includes a pair of shells 211, 212. An end of the shell 212 is sleeved on an end of the shell 211, and the two cooperatively defines an internal space 21a. The adjustment mechanism is configured to adjust an overlap degree between the pair of shells 211, 212 under control of a control unit (for example, a controller), and thereby adjusting a width of the pad 21.

When the pad 21 is in a state shown in FIG. 13A, the overlap degree dl of the pair of shells 211, 212 is less, and a width w1 of the pad 21 is greater. When the pad 21 is in a state shown in FIG. 13B, the overlap degree d2 of the pair of shells 211, 212 is greater, and the width w2 of the pad 21 is less.

In this manner, the adjustment mechanism is able to adjust the width of the pad under the control of the control unit. This implementation has many advantages, such as such as it is relatively simple to implement, and has a compact structure and high reliability.

In some embodiments, the pad 21 may also include an expandable and contractible membrane layer (not shown in the figures) covering the pair of shells 211, 212. The membrane layer may have a smooth outer surface to prevent or reduce proliferation of epidermal cells or other tissues on the exterior of the pad 21.

There are various manners to adjust the overlap degree of the pair of shells 211, 212, which are not specifically limited in the present disclosure.

As an example, referring to FIG. 13A and FIG. 13B, the adjustment mechanism may include a motor 213, a pair of meshing gears 214, 215, a shaft 216 supporting the gear 215 and a rack 217 meshing with the gear 215. The gear 214 is mounted on an output shaft of the motor 213, the shaft 216 is fixed to the shell 211, and an end of the rack 217 is fixed to the shell 212. The motor 213 is able to rotate under control of the control unit to drive the rack 217 to move, and thereby changing the overlap degree of the pair of shells 211, 212.

It may be understood that, the manner of cooperation between the pad and the pair of leaflets is not limited to the above implementation. In the following, an alternative implementation is provided.

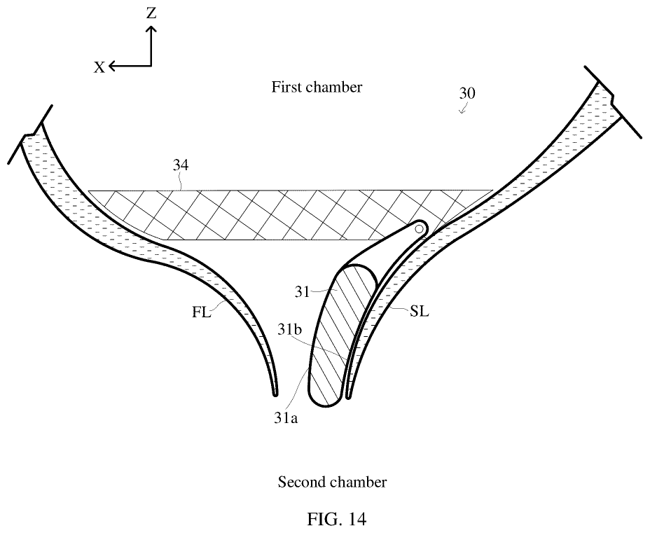

FIG. 14 is a schematic structural diagram showing at least a portion of a medical device 30 according to yet another embodiment of the present disclosure. FIG. 14 illustrates a state that the medical device 30 is placed at a valve of a patient.

The medical device 30 has many same or similar elements with the medical device 10. For purpose of conciseness, the related descriptions are appropriately omitted. It may be understood that, on the premise that no contradiction occurs, elements of the medical device 10 and the medical device 30 may be combined with each other.

Referring to FIG. 14, the medical device 30 includes a pad 31 having a first coaptation surface 31a and a second coaptation surface 31b. When the medical device 30 is implanted at the patient's heart valves, the pad 31 is placed between the pair of leaflets FL, SL. The pad 31 is configured to follow the movement of the second leaflet SL, the second coaptation surface 31b is configured to face and maintain coaptating the second leaflet SL, and the first coaptation surface 31a is configured to face the first leaflet FL and periodically coaptate and separate from the first leaflet FL with the pad 31 following the movement of the second leaflet SL. In other words, during the changes of the cardiac cycle, the second coaptation surface 11b maintains constantly coaptating the second leaflet SL, and the first coaptation surface 11a periodically coaptates and separates from the first leaflet FL.

There are various ways to position the pad between the pair of leaflets to make the pad 31 move with the second leaflet, which is not specifically limited in the present disclosure. Several possible implementations are described below.

As an example, referring again to FIG. 14, the medical device 30 may also include a support member 34. When the medical device 30 is implanted in the patient's heart, the support member 34 is located in the first chamber, and the pad 31 is connected to the support member 34 in a manner allowing it to follow the movement of the second leaflet SL. For instance, the pad 31 may be hinged with the support member 34 to enable the pad 31 to follow the movement of the second leaflet SL. Alternatively, the pad 31 may be connected to the support member via a flexible member (or portion) to make the pad 31 move with the second leaflet SL.

Through the support member placed in the first chamber, the pad is able be reliably positioned between the first and second leaflets. The pad is connected to the support member in a manner enabling it to follow the movement of the second leaflet to ensure that as the second leaflet moves, the pad moves with the second leaflet, so that the first coaptation surface periodically coaptates and separates from the first leaflet.

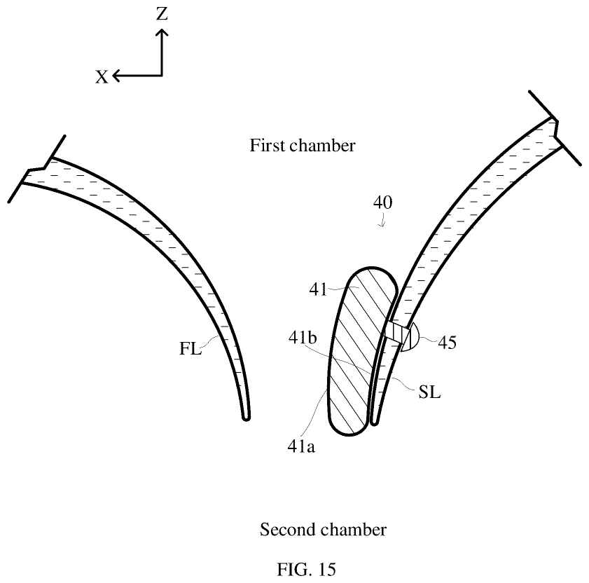

FIG. 15 is a schematic structural diagram showing at least a portion of a medical device 40 according to still another embodiment of the present disclosure. FIG. 15 illustrates a state that the medical device 40 is implanted at a heart valve of a patient.

The medical device 40 has many same or similar elements with the medical devices 10, 30. For the purpose of conciseness, related descriptions are appropriately omitted. It may be understood that, on the premise that no contradiction occurs, elements of the medical device 40 and the medical device 10, 30 may be combined with each other.

As another embodiment shown in FIG. 15, the medical device 40 may include not only a pad 41 but also a pin 45. When the medical device 40 is implanted in the patient's heart, the pip 45 penetrates through the second leaflet SL to attach the pad 41 to the second leaflet SL, and thereby locating the pad 41 between the pair of leaflets FL, SL while enabling it to follow the movement of the second leaflet SL. In this way, with the change of the cardiac cycle, the second coaptation surface 41b facing the second leaflet SL maintains coaptating the second leaflet SL, and the first coaptation surface 41a facing the first leaflet FL periodically coaptates and separates from the first leaflet FL with the pad 41 following with the movement of the second leaflet SL.

The pin may be integrally formed with the pad, or the two may also be two independent members, which is not specifically limited in the present disclosure.

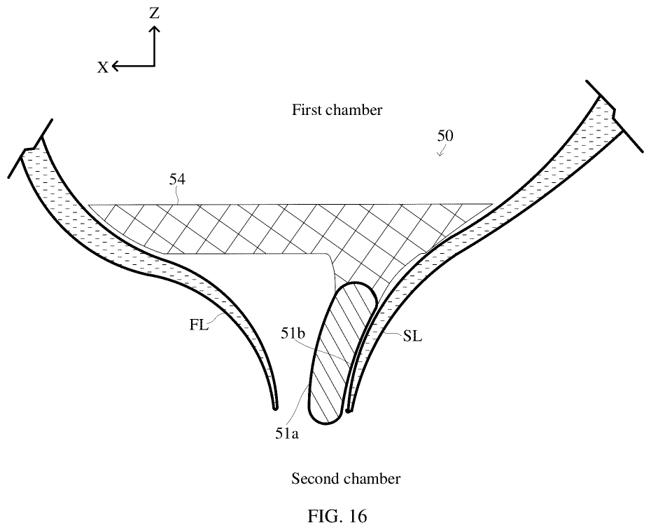

FIG. 16 is a schematic structural diagram showing at least a portion of a medical device 50 according to further another embodiment of the present disclosure. FIG. 16 illustrates a state that the medical device 50 is implanted at a heart valve of a patient.

The medical device 50 has many same or similar elements with the medical devices 10, 30, 40. For the purpose of conciseness, related descriptions are appropriately omitted. It may be understood that, on the premise that no contradiction occurs, elements of the medical device 50 and the medical device 10, 30 and 40 may be combined with each other.

Referring to FIG. 16, the medical device 50 includes a pad 51 and a support member 54. The pad 51 has a first coaptation surface 51a and a second coaptation surface 51b which are configured to face the first leaflet FL and second leaflet SL respectively.

When the medical device 50 is implanted in the patient's heart, the pad 51 is rigidly connected to the support member 54 (that is, connected in a manner preventing relative movement), and thereby maintaining the pad 51 at a preset position between the leaflets FL, SL and keeping the second coaptation surface 51b coaptating the second leaflet SL. This preset position is configured such that the first coaptation surface 51a is able to periodically coaptate and separate from the first leaflet FL with the movement of the first leaflet FL.

In this implementation, since the pad 51 is maintained at the preset position between the leaflets FL, SL with the second coaptation surface 51b coaptating the second leaflet SL, so that the pad 51 will obstruct the movement of the second leaflet SL, and thus the second leaflet SL loses its native physiological function.

Compared with the aforementioned implementations, if the medical device according to this implementation is adopted, after a long service time, the structure and function of the second leaflet may be adversely affected.

In the related art, a current implant, including its support structure, often affects the structure of an existing valve while repairing and improving closure of the valve, and thereby resulting in some hemodynamics and/or clinical issues. After the implant being implanted into the patient's heart, the patient usually needs to go to the hospital regularly for follow-up examination. In general, there are two aspects of purposes of a regular follow-up examination. On one aspect, it is to observe the effect of the implant on the cardiac function (whether it has improved, and the like) and the operating condition of the implant to determine whether there are any adverse events associated with the implant. On the other hand, it is to check the overall condition of the patient, such as heart failure and the use of the medicine, and the like, so that it is determined whether the condition of the patient is effectively improved or whether further deterioration occurs. These follow-up examinations are generally required to be performed in the hospital (or clinic). Frequent follow-up examinations, especially for a patient in remote areas, will cause the economic burden and time cost of the patient to be increased. At the same time, since the follow-up time has a relatively long interval, the change of the condition is often unpredictable, and thereby making it more difficult to achieve the goals of identifying problems early, preventing the condition from worsening and improving the quality of life for patients.

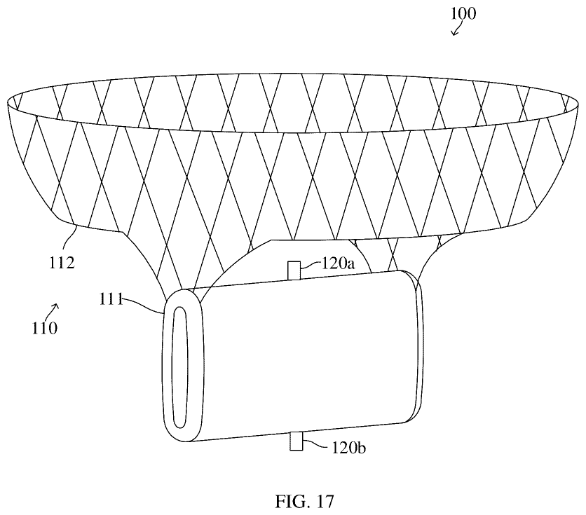

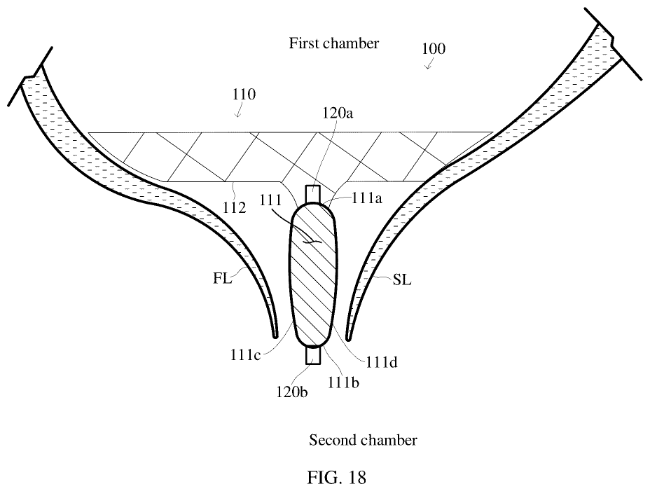

To solve the aforementioned problems, the embodiments of the present disclosure provide a cardiac implant. A cardiac implant 100 according to an embodiment of the present disclosure will be described with reference to FIGS. 17 and 18 below. FIG. 17 is a schematic structural diagram of the cardiac implant 100, and FIG. 18 illustrates a schematic structural diagram of the cardiac implant 100 when it is implanted at a patient's heart valve.

It should be noted that, in the drawings of the present disclosure, the valve is located between a first chamber and a second chamber. The valve periodically allows blood to flow from the first chamber to the second chamber while preventing flowing in the reverse direction. The valve includes a pair of leaflets FL, SL match with each other.

Specifically, the valve may be a mitral valve. Correspondingly, the first chamber may be the left atrium, the second chamber may be the left ventricle, and one of the pair of leaflets FL, SL is the anterior leaflet and the other is the posterior leaflet. It may be understood that, the cardiac implant provided by the present disclosure is not limited to the application in mitral valve, but may also be applied to other heart valve (for example, a tricuspid valve), in this case, the heart valve includes three leaflets.

Referring to FIG. 17 and FIG. 18, the cardiac implant 100 (a medical device) includes a treatment device 110 and a pair of sensors 120a, 120b. It may be understood that, in other examples, the cardiac implant 100 may include only one sensor or three or more sensors. The treatment device 110 is configured to repair a function of the valve to prevent blood from flowing from the second chamber back to the first chamber. The implementation of the treatment device 110 will be described in detail below. The pair of sensors 120a, 120b are attached to the treatment device 110 and are configured to sense physiological information of the patient.

In an example, the physiological information may include, but is not limited to, one or more type of the following: blood pressure information, blood flow velocity information, blood pH information, blood temperature information, blood oxygen level information, electrocardiographic information, heart sound information, cardiac acceleration information and cardiac contractility information of the patient's heart. Correspondingly, the sensors in the present disclosure may include any sensing device that is capable of sensing one or more type of the above-mentioned information.

The cardiac implant provided by the present disclosure is not only able to improve the regurgitation of the patient for realizing a therapeutic purpose, but also is able to sense the physiological information of the heart of the patient for realizing a monitoring purpose. By using the cardiac implant provided by the present disclosure, the patients may need to visit the hospital for follow-ups less frequently or not at all. Consequently, the cardiac implant provided by the present disclosure is able to effectively reduce the financial burden and time cost for the patient. Furthermore, in the present disclosure, at least one sensor is positioned in the patient's heart by attaching to the treatment device rather than being directly anchored to a tissue of the heart of the patient, and thereby harm to the tissue is reduced.

Referring again to FIG. 17 and FIG. 18, the treatment device 110 includes a pad 111. When the cardiac implant 100 is implanted in the patient's heart, the pad 111 is configured to be located between the pair of leaflets FL, SL. The pad 111 is configured to cooperate with the pair of leaflets FL, SL to enable the valve to be periodically opened and closed, and thereby allowing the blood to flow from the first chamber to the second chamber while preventing flowing in the reverse direction. Certainly, in an applications scenario of the tricuspid valve, the pad 111 may be configured to be located among three leaflets. A pair of collection devices (for example, sensors) 120a, 120b are attached to the pad 110 to facilitate fixing of the pair of collection devices 120a, 120b.

Referring again to FIG. 17 and FIG. 18, the pad 111 includes a top surface 111a facing the first chamber and a bottom surface 111b facing the second chamber. The pair of sensors 120a and 120b may be a pair of pressure sensors 120a, 120b. The pair of pressure sensors 120a, 120b may be respectively attached to the top surface 111a and bottom surface 111b of the pad 111 to sense blood pressure information of the first chamber and the second chamber, respectively. In an example, sensing surfaces of the pressure sensor 120a and the pressure sensor 120b may face the first chamber and the second chamber, respectively. The sensing surface may include a vibrating diaphragm.

Sensing surfaces (for example, vibrating diaphragms) of the pair of pressure sensors 120a, 120b faces the first chamber and the second chamber, respectively, and are able to sense blood pressures in the first chamber and the second chamber of the entire cardiac cycle, respectively. This provides the doctor with hemodynamic information having diagnostic value. This information is critical for evaluating the overall cardiac function and the working condition of the valve and the cardiac implant 100. Meanwhile, the pressure information is also able to assist the doctor in evaluating the placement position of the implant during the implantation process and making real-time assessment of whether there is any obstruction to the blood flow into the second chamber during diastole. Therefore, the success of the implantation operation is able to be better ensured, and a better clinical effect is achieved.

Additionally, when the valve opens, the blood flow that flows from the first chamber to the second chamber is able to nearly directly impact onto the pressure sensor 120a (on its vibrating diaphragm) attached to the top surface 111a. This allows the physiological information (that is, blood pressure information) sensed by the pressure sensor 120a to reflect whether the blood flow that flows from the first chamber to the second chamber is normal, so that it is able to relatively accurately reflect whether there is a stenosis issue with the valve of the patient after the cardiac implant 100 is implanted.

When the valve closes, if there is still the regurgitation that flows from the second chamber to the first chamber, the regurgitation will nearly directly impact onto the vibrating diaphragm of the pressure sensor 120b attached to the bottom surface 111b, so that the physiological information (that is, pressure information) and variations thereof sensed by the pressure sensor 120b due to the blood flow will reflect whether there is the regurgitation that flows from the second chamber to the first chamber.

It may be understood that, when the pressure sensor is configured to side face sideways to the first chamber and second chamber, the blood flow will impact the sensing surface of the pressure sensor from the side, so that the pressure response generated by the pressure sensor will be lower than the actual pressure of the blood flow. Conversely, when the pressure sensor is configured to face the first chamber and second chamber, the blood flow will directly impact onto the sensing surface, so that the pressure sensor is able to more accurately reflect the actual pressure of the blood flow.

Furthermore, as shown in FIG. 17 and FIG. 18, the pair of sensors 120a, 120b are located at the middle position of the top surface 111a and bottom surface 111b of the pad 111 in the width and thickness directions of the pad, respectively. When the pair of pressure sensors 120a, 120b are arranged centrally on the pad 111 located between the leaflets of the valve, they are located at the central position of the blood flow path, at this position they are able to more accurately reflect the overall pressure of the atrium or ventricle, and thereby more accurately reflecting whether there is stenosis or regurgitation at the valves.

On the other hand, as illustrated in FIG. 18, since the pair of pressure sensors 120a, 120b are attached to the top surface 111a and bottom surface 111b of the pad 111 respectively, while the leaflets of the valve are located on the sides of the pad 111, therefore, the top surface 111a and the bottom surface 111b of the pad will not contact with the leaflets of the valve when the valve closes. Consequently, the leaflets are prevented from interfering with the pressure sensors 120a, 120b, so that the accuracy of the pressure sensors 120a, 120b sensing the blood pressure is improved, and thus whether there is stenosis or regurgitation at the valve is more accurately and more timely reflected.

Consequently, by using the pair of pressure sensors 120a, 120b respectively attached to the top surface 111a and the bottom surface 111b of the pad 111, whether the valve has stenosis or regurgitation issues after the cardiac implant 100 is implanted is able to be reliably detected, so that a more accurate assessment of the patient's postoperative recovery and development of the condition of the patient is able to be accurately monitored.

It should be noted that, although in the foregoing embodiments, the cardiac implant 100 includes the pair of sensors 120a, 120b, in other implementations, the cardiac implant 100 may include merely the sensor 120a attached to the top surface 111a, or may include merely the sensor 120b attached to the bottom surface 111b. Certainly, in other examples, the cardiac implant 100 may include more sensors.

It should also be noted that, although in the foregoing embodiment, the pair of sensors 120a, 120b are respectively attached to the top surface 111a and the bottom surface 111b of the pad 111, in other examples, the pair of sensors 120a, 120b may also be attached to other portions of the pad 111. For example, in some examples, one or more sensors may be attached to the sides of pad 111. In other examples, for example for a pad having the structure shown in FIG. 7, one or more sensors may be attached to a portion of the top surface 512 of the pad 111 close to the first coaptation surface 511 such that the sensor is located in an intermediate position of the two leaflets.

Continuing referring to FIG. 17 and FIG. 18, the pad 111 further includes a pair of coaptation surfaces 111c, 111d. The pair of coaptation surfaces 111c, 111d are configured to face the pair of leaflets FL, SL, respectively. As the cardiac cycle changes, each coaptation surface of the pair of coaptation surfaces 111c, 111d periodically coaptates and separates from the leaflet it faces, and thereby enabling the valve to be periodically opened and closed. Specifically, when one of the coaptation surfaces 111c, 111d coaptates its corresponding leaflet, a coaptation length between them may range from 6 mm to 12 mm. That is, a length of the coaptation surfaces 111c, 111d may also range from 6 mm to 12 mm. In particular, the leaflet FL may be the anterior leaflet, and the leaflet SL may be the posterior leaflet, and the pad 111 is configured to be closer to the leaflet SL than to the leaflet FL. Specifically, the coaptation surfaces 111c, 111d may be smooth transition curved surfaces to reduce resistance to the blood flow.

When the second chamber is in the diastole, the pair of leaflets FL, SL move away from each other to form passages between the pair of leaflets FL, SL and the pair of coaptation surfaces 111c, 111d, respectively. At this time, the valve opens to allow the blood flow to pass through these passages from the first chamber into the second chamber. When the second chamber is in the systole, the pair of leaflets FL, SL move toward each other to make the pair of leaflets FL, SL coaptate the pair of coaptation surfaces 111c, 111d, respectively. At this time, the valve closes to prevent the blood flow flowing from the second chamber back to the first chamber. It should be noted that, in the present disclosure, when a leaflet coaptates a coaptation surface, the leaflet and the coaptation surface may be in contact with each other to prevent the blood flow from flowing therebetween.

In this implementation, the pad 111 does not affect or minimally affects the movement of the pair of leaflets FL, SL, so that the pair of leaflets FL, SL are able to maintain their original physiological functions. That is, in this implementation, as the cardiac cycle changes, the pair of leaflets FL, SL are able to naturally and periodically move toward and away from each other. Therefore, by using the treatment device according to this implementation, there will be minimally or even no adverse effects on the structure and function of the pair of leaflets FL, SL. In other words, the cardiac implant 100 provided by the present disclosure has no restriction or hindrance on the natural movement (closing, opening) of the valve, and it is not associated with the supporting structure of the valve, so that the corresponding adverse effect is avoided.

Continuing referring to FIG. 17 and FIG. 18, the treatment device 110 may also include a support member 112. After the cardiac implant 100 is implanted into a patient's heart, the support member 112 is located in the first chamber and is connected to the pad 111, and thereby positioning the pad 111 between the pair of leaflets SL, FL. By means of the support member 112 located in the first chamber, the pad 111 is able to be reliably positioned between the pair of leaflets FL, SL. In particular, the connection position between the support member 112 and the pad 111 is configured to be adjustable to allow the position of the pad between FL and SL to be adjusted.

Continuing referring to FIG. 17 and FIG. 18, the support member 112 may be annular in shape, and the pad 111 is centrally disposed on a side of the support member 112 along a first radial direction of the support member 112, such that the pad 111 is centrally positioned between the pair of leaflets FL, SL of the valve. The length direction of the pad 111 is consistent with the first radial direction, and the width direction and height direction of the pad 111 are perpendicular to the first radial direction. Along the length direction of the pad 211, the pad 211 has two side surfaces which are arc-shaped curved surfaces configured to fit with the leaflets. After the cardiac implant 100 is implanted into the patient's heart, the support member 112 may be placed at the valve annulus of the heart. In some embodiments, the support member 112 may have a mesh frame structure. During delivery, the support member 112 may firstly be folded to reduce its volume. After being delivered to a target position, it may be unfolded again.

It may be understood that, the implementation of the support member 112 is not limited to the above, as long as it is able to position the pad 111 between the pair of leaflets FL, SL of the valve. For example, in some embodiments, the support member 112 may also be semi-annular or partially annular in shape.

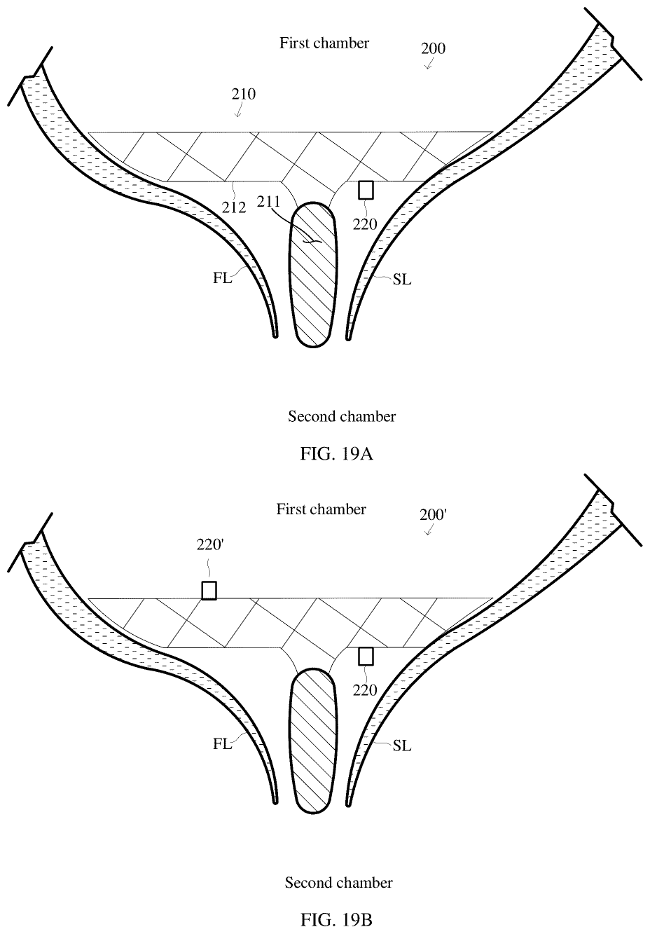

FIG. 19A is a schematic structural diagram illustrating a cardiac implant 200 according to another embodiment of the present disclosure that is implanted in a patient's heart. The cardiac implant 200 is substantially the same as the cardiac implant 100. For the sake of brevity, the similarities will not be repeated herein.

Referring to FIG. 19A, the cardiac implant 200 includes a treatment device 210. The treatment device 210 includes a pad 211 and a support member 212. The pad 211 is substantially the same as the pad 111 in the aforementioned embodiment, and the support member 212 is substantially the same as the support member 112 in the aforementioned embodiment. Relevant descriptions of the pad 211 and the support member 212 may refer to the descriptions of the pad 111 and the support member 112 in the aforementioned embodiments.

The cardiac implant 200 also includes a sensor 220. The sensor 220 is attached to the support member 212 and is configured to sense physiological information of the patient. In particular, the sensor 220 may be a pressure sensor. Specifically, the sensor 220 may be attached to a portion of the support member 212 closer to the body surface, such as closer to the anterior, left, or posterior chest wall. In particular, the sensor 220 may be attached to the support member 212, and their relative positions may be configured such that the support member 212 is not located between the sensor 220 (at least the communication portion of the sensor 220) and an external device in communication with the sensor 220. In this way, interference from the support member 212 (especially when it is made of metal) on wireless communication between the external device and the sensor is minimized.

As shown in FIG. 19A, the support member 212 has an upper side and a lower side along a direction from the first chamber to the second chamber. The upper side is farther from the pad 211 and closer to the first chamber compared to the lower side, and the lower side is closer to the pad 211 and closer to the second chamber compared to the upper side. An area between the upper side and the lower side is an intermediate region of the support member 212. Both the sensor 220 and the pad 211 are located at the lower side that closer to the second chamber, and the sensor 220 is offset from the pad 211 in the radial direction of the support member 212. It may be understood that, the support member 212 (or its main body portion) is generally annular (closed or unclosed) or has a circular surface facing the second chamber. Therefore, the orientation term “radial” in the present disclosure may be relative to this annular or circle.

Specifically, referring to FIG. 19A, the sensor 220 and the pad 211 are offset from each other along the direction from one leaflet SL to the other leaflet FL, such that when the support member 212 is implanted between the leaflets of the valve, the sensor 220 is located between the leaflet SL and the pad 211. The sensor 220 is a pressure sensor, and an end of the sensor 220 is fixed to a lower edge or a lower surface of the support member 212 (that is, the edge or surface on the lower side of the support member 212), and the other end of the sensor 220 faces the second chamber, so that this facilitates sensing the pressure of blood flow that flows from the second chamber into the first chamber.

It may be understood that, the other end of the sensor 220 has a sensing surface. When the valve is closed, the sensing surface is located in a region formed by the lower side of the support member 212, the leaflet SL and the pad 211 and faces the second chamber. This ensures that if regurgitation that flows from the second chamber to the first chamber exists between the leaflet SL and the pad 211, the regurgitant will nearly directly impact on to the sensing surface of the sensor 220, so that the sensor 220 detects pressure information due to the blood flow. By arranging the sensor 220 on the support member 212, between the leaflet SL and the pad and the sensing surface facing toward the second chamber, the sensor is able to quickly and accurately sense the blood pressure caused by the regurgitation. If the sensor is set to be in other orientations (for example, facing the first chamber), it might not be able to, or would more difficult/slower to detect the regurgitation from the second chamber due to pressure and directional changes in blood flow. In the present embodiment, however, even there is minimal regurgitation between the first and second chamber, the sensor 220 may sensitively capture it.

It should be understood that, although merely one sensor 220 is shown in FIG. 19A, the cardiac implant 200 may include a plurality of sensors 220. The plurality of sensors 220 may be attached to different portions of the support member 212. For example, one sensor 220 may be disposed at a lower edge of the support member 212 and between the leaflet FL and the pad 211, and another sensor 220 may be disposed at the lower edge of the support member 212 and between the leaflet SL and the pad 211. In the case that the valve is a tricuspid valve, there may be one sensor 220 disposed at the lower edge of the support member 212 and between the third leaflet and the pad 211. Alternatively, some of the plurality of sensors 220 may be attached to the support member 212, and others may be attached to the pad 211.

In other examples, the sensor 220 may also be fixed to the intermediate region of the support member 212, and a sensing surface of the sensor 220 faces the second chamber. In this case, a portion of the sensor 220 may be located in the intermediate region of the support member 212, and another portion extends beyond the lower side of the support member 212, or the entire sensor 220 may be located at the intermediate region of the support member 212. When the support member is an annular structure, the sensor 220 may also be located on an inner surface of the support member 212. Compared to fixing the sensor 220 to the edge or surface of the lower side, fixing it to the intermediate region of the support member 212 is easier to implement and facilitates a stable connection between the sensor 220 and the support member 212.

FIG. 19B is a schematic structural diagram illustrating a cardiac implant 200′ according to another embodiment of the present disclosure that is implanted in a patient's heart. The cardiac implant 200′ is substantially the same as the cardiac implant 200. For the sake of brevity, the similarities will not be repeated herein.

As shown in FIG. 19B, the sensor 220′ may be fixed to the intermediate region of the support member 212, or to an edge or surface of the upper side, or to an inner surface. Meanwhile, an end of the sensor 220′ has a sensing surface configured to face the first chamber to sense a blood pressure in the first chamber. It may be understood that, in this case, the sensor 220′ faces the first chamber and faces away from the second chamber, and therefore it is not subject to direct impact from the regurgitation. Compared to the blood pressure sensed by sensor 220, the blood pressure sensed by sensor 220′ is more stable, and is able to more accurately reflect the overall pressure condition in the first chamber. Both sensor 220 and sensor 220′ may be simultaneously installed on the support member 212 to provide pressure information from different positions, so that the doctor is able to make more precise assessment of the patient's valve status, or the control unit shown in FIG. 12A is able to control the adjustment mechanism to make more appropriate adjustments.

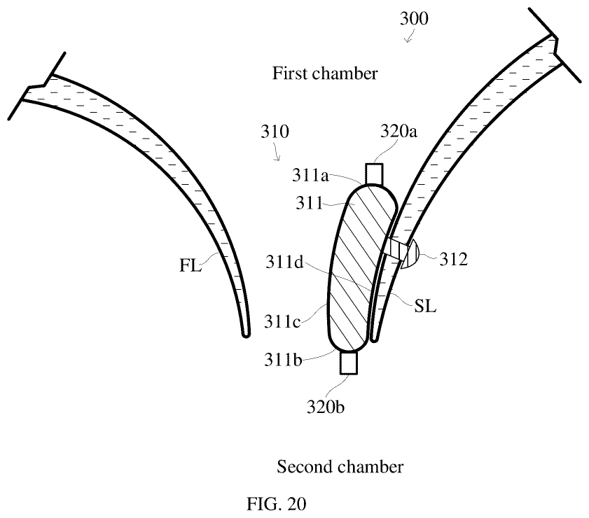

FIG. 20 is a schematic structural diagram illustrating a cardiac implant 300 according to another embodiment of the present disclosure that is implanted in a patient's heart. The cardiac implant 300 is substantially the same as the cardiac implant 100. For the sake of brevity, the similarities will not be repeated.

Referring to FIG. 20, the cardiac implant 300 includes a treatment device 310 and a pair of sensors 320a, 320b. The treatment device 310 includes a pad 311 having a top surface 311a and a bottom surface 311b that face the first chamber and the second chamber, respectively. The pair of sensors 320a,320b may be a pair of pressure sensors 320a, 320b, and the pair of pressure sensors 320a, 320b may be attached to the top surface 311a and bottom surface 311b, respectively.

It should be noted that, in other examples, the cardiac implant 300 may include merely one of the pair of sensors 320a, 320b. It should also be noted that, in other examples, the sensors of cardiac implant 300 may be attached to positions on a place other than the top surface 311a and bottom surface 311b, such as be attached to the support frame. It should also be noted that, in other examples, the sensors of cardiac implant 300 may be sensors that are capable of sensing other physiological information.

The cooperation mode between the pad 311 and the pair of leaflets FL, SL differs from that between the pad 111 and the pair of leaflets FL, SL in the aforementioned embodiments. Specifically, the pad 311 includes a pair of coaptation surfaces 311c, 311d. After the cardiac implant 300 is implanted into the patient's heart, the pad 311 is configured to be located between the pair of leaflets FL, SL and follow the movement of leaflet SL, and the pair of coaptation surfaces 311c, 311d are configured to respectively face the pair of leaflets FL, SL. As the pad 311 follows the movement of leaflet SL, the coaptation surface 311d is configured to maintain coaptating the leaflet SL (for example, a coaptation length between them may range from 6 mm to 12 mm), and the coaptation surface 311c is configured to periodically coaptate and separate from leaflet FL. In other words, with changes in the cardiac cycle, the coaptation surface 311d remains in constant coaptate the leaflet SL, while the coaptation surface 311c periodically coaptates and separates from the leaflet FL.

In this implementation, the pad 311 does not affect or minimally affects the movement of the pair of leaflets FL, SL, so that the pair of leaflets FL, SL, particularly the leaflet FL, are able to maintain their original physiological functions. That is, in this implementation, as the cardiac cycle changes, the pair of leaflets FL, SL are able to naturally and periodically move toward and away from each other. Consequently, it may be seen that, this implementation has minimal adverse effects on the structure and function of the leaflets FL, SL.

There are many ways to position the pad 311 between the pair of leaflets FL, SL and to make the pad 311 move with the leaflet SL, which is not specifically limited in the present disclosure. As an example, continuing referring to FIG. 20, the treatment device 310 also includes a pin 312 or other fixing member, this kind of fixing member is connected to the pad 311 and used for connecting the pad 311 to the leaflet. Specifically, the pin 312 may penetrate through the leaflet SL to attach the pad 311 to leaflet SL, and thereby locating the pad 311 between the pair of leaflets FL, SL and enabling it to follow the movement of leaflet SL. In this way, as the cardiac cycle changes, the coaptation surface 311d facing leaflet SL is able to maintain coaptating the leaflet SL, and the coaptation surface 311c facing leaflet FL is able to periodically coaptate and separate from the leaflet FL with the pad 311 following the movement of leaflet SL.

The pin 312 may be integrally formed with the pad 311, and the two may also be two independent members, which are not specifically limited in the embodiments of the present disclosure.

Although the pad 311 in FIG. 20 is disposed on the leaflet on a side, the top surface 311a and bottom surface 311d of the pad 311 still face the first chamber and the second chamber, respectively. Consequently, the pressure sensors 320a, 320b on the top surface 311a and bottom surface 311d are also disposed to face the first chamber and the second chamber, respectively. Therefore, the blood flow will directly impact onto the pressure sensors 320a, 320b, so that the pressure sensors 320a, 320b are able to accurately reflect the pressure of the blood flow.

As shown in FIG. 20, since the sensors 320a, 320b are attached to the top surface 311a and bottom surface 311b of the pad 311, respectively, the sensor 320a is away from the leaflet SL in a manner facing the first chamber, and the sensing surface of sensor 320b is away from a distal end of the leaflet SL in a manner facing the second chamber, so that the pressure sensors 320a, 320b will not contact with the leaflets of the valve when the valve closes. This prevents the valve from interfering with the pressure sensors 320a, 320b, and thereby the accuracy of the blood pressure sensed by the pressure sensors 320a, 320b is further improved.

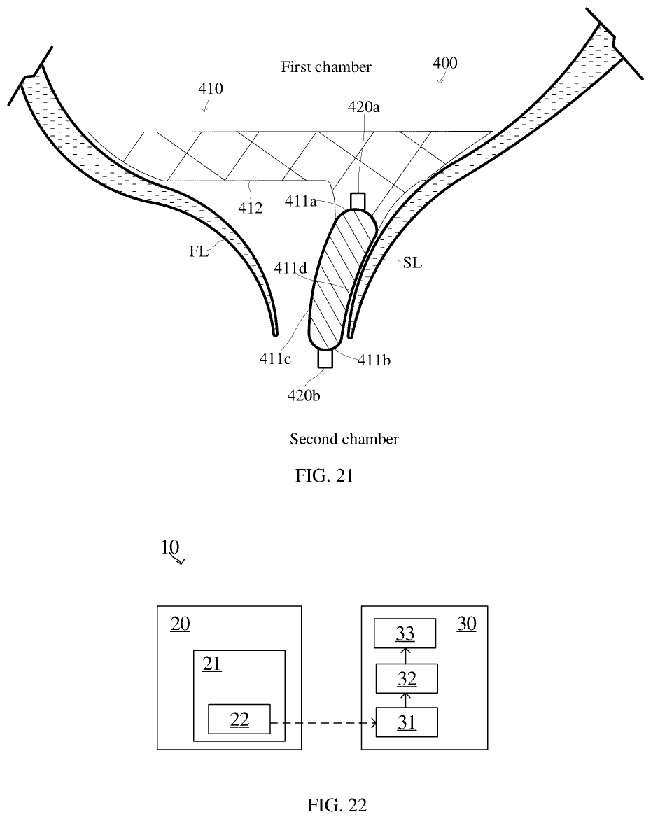

FIG. 21 is a schematic structural diagram illustrating a cardiac implant 400 according to another embodiment of the present disclosure that is implanted in a patient's heart. The cardiac implant 400 is substantially the same as the cardiac implant 100. For the sake of brevity, the similarities will not be repeated.

Referring to FIG. 21, the cardiac implant 400 includes a treatment device 410 and a pair of sensors 420a, 420b. The treatment device 410 includes a pad 411 and a support member 412 for positioning the pad 411 between the pair of leaflets FL, SL. The support member 412 is substantially the same as the support member 112 in the aforementioned embodiments. The pad 411 has a top surface 411a facing the first chamber and a bottom surface 411b facing the second chamber. The pair of sensors 420a, 420b may be a pair of pressure sensors 420a, 420b, which may be respectively attached to the top surface 411a and bottom surface 411b. The difference lies in that the pad 411 is offset to a side of the support member 412, and in addition, the connection between the support member 412 and the pad 411 is configured to be movable to allow the pad 411 to move with leaflet SL during the cardiac cycle.

It should be noted that, in other examples, the cardiac implant 400 may include merely one of the pair of sensors 420a, 420b. It should also be noted that, in other examples, the sensors of the cardiac implant 400 may be attached to positions on pad 411 other than the top surface 411a and bottom surface 411b. For instance, in some examples, the cardiac implant 400 may also include at least one sensor attached to the support member 412, and the t least one sensor is disposed at a lower edge of the support member 412 and faces the second chamber, and radially offset from the pad 411 and disposed between the pad 411 and the leaflet FL. It should be noted that, in other examples, the sensors of cardiac implant 400 may also be sensors that are capable of sensing other physiological information.

The pad 411 also includes a pair of coaptation surfaces 411c, 411d. The pair of coaptation surfaces 411c, 411d are configured to respectively face the pair of leaflets FL, SL. After the cardiac implant 400 is implanted into the patient's heart, the pad 411 is rigidly connected to the support member 412 (that is, connected in a manner that prevents relative movement), and thereby maintaining the pad 411 at a preset position between the pair of leaflets FL, SL while keeping the coaptation surface 411d coaptating the leaflet SL. This preset position is configured such that the coaptation surface 411c is able to periodically coaptate and separate from the leaflet FL with the movement of the leaflet FL, and the leaflet FL coaptates the coaptation surface 411c during the systole and achieves a certain coaptation length (for example, 6 mm˜12 mm), so that effective closure of leaflets is increased, and thus the blood flow that flows from the second chamber to the first chamber is prevented. During diastole, the leaflet FL opens to allow the blood to flow from the first chamber through a passage between leaflet FL and the coaptation surface 411c into the second chamber. In particular, the pad 411 is configured to avoid causing stenosis during diastole due to an insufficient space between the leaflet FL and the coaptation surface 411c, such as by adjusting the thickness of pad 411 and/or its position relative to the leaflet FL.

In another embodiment of the present invention, a cardiac implant system 10 is also provided. Referring to FIG. 22, the implant system 10 includes a cardiac implant 20 and an external device 30. The cardiac implant 20 may be any one of the cardiac implants 100, 200, 300 or 400 described in the foregoing embodiments. The sensor 21 of the cardiac implant 20 has a wireless transmission unit 22 (for example, a transmission interface or a signal transmitter). The external device 30 includes a wireless reception unit 31, an analysis unit 32 and an alarm unit 33 (for example, a buzzer, a flashing light, and the like) that are in communication connection with each other. The wireless reception unit 31 receives pressure value information from the wireless transmission unit 22 of the sensor 21. The analysis unit 32 (for example, a controller, a monitor or a terminal device has an analysis function, such as a computer, a mobile phone, and the like) determines whether to send an alarm signal to the alarm unit 33 based on the pressure value information. Upon receiving the alarm signal, the alarm unit 33 issues an alarm message through any one or a combination of a pattern, text or sound to alert the doctor with the abnormal opening/closing conditions of the valve. Consequently, the doctor is able to track and determine the post-implantation conditions of the implant 20 based on the sensed pressure value information and promptly carry out the subsequent treatment.

Specifically, for the cardiac implants 100, 200, 300, 400 or 500, it may be configured such that when a pressure difference between the two sensors 120a, 120b (or between two sensors 320a, 320b, or between two sensors 420a, 420b, or between two sensors 520a, 520b) exceeds a preset threshold (for example, 5 mmHg) within a specified time period (indicating possible valve stenosis), the analysis unit 32 send an alarm signal to the alarm unit 33. For the cardiac implant 200, it may be configured such that when the change in the pressure value is greater than a preset threshold within a specified time period (indicating possible valve regurgitation), the analysis unit 32 send an alarm signal to the alarm unit 33. This predetermined threshold may be determined based on pressure values obtained under normal valve closure conditions.

It should be understood that, although terms such as “first” or “second” may be used in the present disclosure for describing various elements (for example, the first chamber and the second chamber), but these elements are not limited by these terms, and these terms are merely used for distinguishing one element from another.

It should be noted that, the various specific technical features (elements) described in the above detailed embodiments may be combined in any suitable manner without contradiction. For example, the pad shown in FIG. 1 to FIG. 10 may has the adjustment structure illustrated in FIG. 12A to FIG. 13B, or may include the sensors depicted in FIG. 11 and FIG. 17 to FIG. 21. To avoid unnecessary repetition, the present disclosure will not be further described in various possible combinations.

It should be understood that, in the embodiments of the present disclosure, “at least one” refers to one or more, and “a plurality of” refers to two or more. The term “and/or” describes the association relationship of associated objects, indicating that three relationships may exist. For example, “A and/or B” may represent a case where A exists alone, both A and B exist, and B exists alone.

It should be understood that, multiple members and/or portions may be provided by a single integrated member or portion. Alternatively, a single integrated member or portion may be divided into separate multiple members and/or portions. The use of “a” or “an” to describe a member or portion is not intended to exclude other members or portions.

The foregoing descriptions are merely specific implementations of the present disclosure, but the protection scope of the present disclosure is not limited thereto. Any person skilled in the art may conceive of modifications or substitutions within the technical scope disclosed in the present disclosure, and such modifications or substitutions should be covered by the protection scope of the present disclosure. Therefore, the protection scope of the present disclosure shall be subject to the scope defined by the claims.

What is claimed is:

1. A medical device for improving a function of a valve of a patient, comprising a pad, the pad being configured to be capable of being positioned among a plurality of leaflets of the valve to make the valve periodically open and close by cooperating with the plurality of leaflets.

2. The medical device according to claim 1, wherein the plurality of leaflets comprises a first leaflet and a second leaflet, the pad has at least a first coaptation surface, the first coaptation surface is configured to face the first leaflet and periodically coaptate and separate from the first leaflet as the first leaflet and/or the second leaflet moves, and the first coaptation surface is a concave surface.

3. The medical device according to claim 2, wherein the valve allows blood to flow from a first chamber to a second chamber and prevent flowing in a reverse direction, along a height direction of the pad, a cross-sectional area of the pad gradually increases from a bottom end to a top end of the pad, and the first coaptation surface is configured to gradually extend toward a side where the first leaflet is located in a direction trending towards the first chamber.

4. The medical device according to claim 3, wherein the first coaptation surface has a first edge portion and a second edge portion respectively located at two opposite ends of the first coaptation surface, the first edge portion is located at the top end of the pad, the second edge portion is located at the bottom end of the pad, and an included angle formed between a tangent plane of the first edge portion and a tangent plane of the second edge portion ranges from 10° to 45°.

5. The medical device according to claim 3, wherein the pad further comprises a second coaptation surface, and the second coaptation surface is configured to face the second leaflet and gradually extend toward a side where the second leaflet is located in the direction trending towards the first chamber.

6. The medical device according to claim 2, wherein the valve allows blood to flow from a first chamber to a second chamber and prevents flowing in a reverse direction, the pad further has a top surface, the top surface is a convex surface configured to face the first chamber, an included angle formed between a tangent plane of a central portion of the top surface and a tangent plane of an edge portion of the top surface ranges from 10° to 45°, and the edge portion of the top surface is close to the leaflet.

7. The medical device according to claim 1, wherein the valve allows blood to flow from a first chamber to a second chamber and prevents flowing in a reverse direction, the medical device further comprises a support member connected to the pad, the support member is configured to be located in the first chamber to position the pad among the plurality of leaflets, the support member has a main body portion and a pair of connecting portions, and the pair of connecting portions are configured to extend to two opposite sides of the pad along a width direction of the pad respectively and be connected to the pad.

8. The medical device according to claim 1, wherein the pad comprises an expandable mesh frame, a cover covering an outer side of the mesh frame and a plurality of coaptation surfaces, and the cover is disposed at a top end of the pad without covering any of the plurality of coaptation surfaces.

9. The medical device according to claim 2, wherein the pad is configured to move following the second leaflet, and the first coaptation surface is configured to periodically coaptate and separate from the first leaflet as the pad moves following the second leaflet; and the medical device further comprises a pressure sensor, the pad further comprises a second coaptation surface for coaptating the second leaflet, and the pressure sensor is disposed on a top surface of the pad and is closer to the first coaptation surface than to the second coaptation surface.

10. The medical device according to claim 9, wherein the pad is configured to be attached to the second leaflet.

11. The medical device according to claim 9, wherein the valve allows blood to flow from a first chamber to a second chamber and prevents flowing in a reverse direction, the medical device further comprises a support member connected to the pad, the support member is configured to be located in the first chamber and position the pad between the first leaflet and the second leaflet, and the pad is connected to the support member in a manner that allows the pad to move following the second leaflet.

12. The medical device according to claim 1, further comprising:
an adjustment mechanism; and
a control unit configured to control the adjustment mechanism to adjust a dimension of the pad.

13. The medical device according to claim 12, further comprising a wake-up unit configured to wake up the control unit based on a preset schedule.

14. The medical device according to claim 12, further comprising a wake-up unit and a sensor, wherein the sensor is configured to sense a physiological information of the patient, and the wake-up unit is configured to wake up the control unit in response to the physiological information satisfying a preset condition.

15. The medical device according to claim 14, further comprising a support member connected to the pad, wherein the support member is configured to be located in the first chamber to position the pad among the plurality of leaflets, the sensor is disposed on a coaptation surface of the pad along a thickness direction of the pad, and along a height direction of the pad, the sensor is closer to an end of the pad connected to the support member than to an end of the pad away from the support member.

16. The medical device according to claim 14, wherein the valve allows blood to flow from a first chamber to a second chamber and prevents flowing in a reverse direction, the plurality of leaflets comprises a first leaflet and a second leaflet, the pad comprises at least a first coaptation surface configured to face the first leaflet and periodically coaptate and separate from the first leaflet as the first leaflet and/or the second leaflet moves, and the sensor is disposed on the coaptation surface and is closer to a top end of the pad than to a bottom end of the pad.

17. The medical device according to claim 12, wherein the pad comprises a pair of main body members, the pair of main body members are stacked along a thickness direction of the pad, each main body member has a free end and a connected end opposite to each other, the connected ends of the pair of main body members are pivotally connected, and the adjustment mechanism is configured to adjust a thickness of the pad by adjusting an opening degree between the pair of main body members under the control of the control unit.

18. The medical device according to claim 12, wherein the pad comprises a pair of shells, an end of one shell is sleeved on an end of the other shell in the width direction of the shells, and the pair of shells cooperatively define an internal space; and the adjustment mechanism is configured to adjust an overlapping degree of the pair of shells under the control of the control unit to adjust a width of the pad.

19. The medical device according to claim 1, adapted to be implanted into a heart, wherein the heart comprises a first chamber and a second chamber, the medical device further comprises at least one sensor, and the at least one sensor is configured to sense a physiological information.

20. The medical device according to claim 19, wherein the at least one sensor is attached to the pad, and the pad and the at least one sensor are configured in at least one of the following manners:
the pad having a top surface configured to face the first chamber, and the at least one sensor comprising a pressure sensor attached to the top surface of the pad to sense a blood pressure in the first chamber;
the pad having a bottom surface configured to face the second chamber, and the at least one sensor comprising a pressure sensor attached to the bottom surface of the pad to sense a blood pressure in the second chamber.

21. The medical device according to claim 19, wherein the pressure sensor is attached to a central position of the top surface of the pad or a central position of the bottom surface of the pad in a width direction and a thickness direction of the pad.

22. The medical device according to claim 19, wherein the medical device further comprises a support member connected to the pad, the at least one sensor comprises a pressure sensor, and the pressure sensor is fixed to the support member.

23. The medical device according to claim 22, wherein the pressure sensor is arranged in one of the following manners:
at least a portion of the pressure sensor being disposed in an intermediate region of the support member;
the pad and the pressure sensor being located on different sides of the support member;
the pad and the pressure sensor being located on a same side of the support member and being staggered in a radial direction of the support member.

24. The medical device according to claim 22, wherein the pressure sensor has a sensing surface, and the sensor is configured in one of the following manners:
the sensing surface being configured to face the first chamber to sense a pressure of blood flow in the first chamber;
the sensing surface being configured to face the second chamber to sense a pressure of blood flow entering the first chamber from the second chamber.

25. An implant system comprising the medical device according to claim 19 and an external device, wherein the at least one sensor comprises a wireless transmission unit, the external device comprises a wireless reception unit for receiving a pressure value information from the wireless transmission unit of the sensor, the external device further comprises an analysis unit and an alarm unit, and the analysis unit receives the pressure value information from the wireless reception unit and determines whether to send an warning signal to the alarm unit according to the pressure value information; and after the alarm unit receives the alarm signal, the alarm unit sends an alarming information.
